(19) United States
(12) Patent Application Publication
(10) Pub. No.: US 2017/0065673 A1
(43) Pub. Date: Mar. 9, 2017

(54) NANOPARTICLE AGGREGATES CONTAINING OSTEOPONTIN AND CALCIUM- AND/OR STRONTIUM-CONTAINING PARTICLES

(71) Applicant: ARLA FOODS AMBA, Viby J (DK)

(72) Inventors: Henrik Birkedal, Lystrup (DK); Jakob OLSEN, Aarhus N (DK); Jonas SKOVGAARD, Aarhus C (DK); Sebastian SCHLAFER, Abyhoj (DK); Rikke Louise MEYER, Abyhoj (DK); Bente NYVAD, Risskov (DK); Duncan Stewart SUTHERLAND, Aarhus V (DK); Peter Langborg WEJSE, Aarhus N (DK)

(21) Appl. No.: 15/091,499

(22) Filed: Apr. 5, 2016

Related U.S. Application Data
(63) Continuation of application No. 14/387,946, filed on Sep. 25, 2014, now abandoned.

(30) Foreign Application Priority Data
Mar. 28, 2012 (EP) 12161802.9

(51) Int. Cl. A61K 38/17

(52) U.S. Cl. CPC A61K 38/1709

(57) ABSTRACT

The present invention relates to nanoparticle aggregates comprising osteopontin (OPN) and one or more particles containing calcium and/or strontium and to their use for reducing or preventing biofilm growth or for removing biofilm. The invention furthermore relates to the use of the nanoparticle aggregates for treating, alleviating or preventing biofilm-related diseases.

### TECHNICAL FIELD OF THE INVENTION

The present invention relates to nanoparticle aggregates comprising osteopontin (OPN) and one or more particles containing calcium and/or strontium and to their use for reducing or preventing microbial biofilm growth or for removing microbial biofilm. The invention furthermore relates to the use of the nanoparticle aggregates for treating, alleviating or preventing biofilm-related diseases.

### BACKGROUND OF THE INVENTION

A vast amount of damage is caused by microbial biofilms. Bacterial and fungal biofilms are involved in numerous human diseases, including bacterial endocarditis, chronic wound infections, implant infections, otitis media, caries, periodontitis and cystic fibrosis. Moreover, biofilms play an important role in food spoilage and biofouling, both of which cause huge economic losses world-wide. While conventional anti-biofilm approaches aim at the mechanical removal of biofilms and/or the killing of bacteria in the biofilms, alternative strategies target the mechanisms involved in microbial biofilm formation (adhesion, coaggregation, biofilm maturation). Still, the harmful effects of microbial biofilms remain a major global problem. Dental caries, for example, is still the most widespread human disease.

WO2005053628 discloses the use of OPN for reducing plaque bacterial growth on tooth enamel and dental formulations containing osteopontin.

Jensen et al. (Journal of Biomedical Material Research A, October 2011, Vol 99A. Issue 1) discloses hydroxyapatite nanoparticles coated with OPN and their use for implant coatings.

Holt et al. (FEBS Journal 276 (2009), pages 2308-2323) discloses the production of calcium phosphate nanoclusters using OPN or OPN fragments for controlling the growth of the calcium phosphate cores.

### SUMMARY OF THE INVENTION

The inventors of the present invention have found that nanoparticle aggregates comprising OPN and a particle comprising calcium surprisingly provide a large reduce oral biofilm growth both in vitro and in vivo (see e.g. Examples 6 and 8, and FIGS. 3, 6A and 6B). The nanoparticle aggregates are highly efficient in reducing biofilm formation, much more so than OPN alone, or calcium-containing particles without OPN, or other model particles. Thus, a clear synergy is obtained by combining OPN and calcium-containing particles leading to improved anti-biofilm effects. Furthermore, the inventors have shown that calcium can be replaced by strontium.

Thus, an aspect of the invention pertains to nanoparticle aggregates comprising a) OPN and b) a first particle comprising calcium and/or strontium, for use as a medicament.

Another aspect of the invention pertains to nanoparticle aggregates comprising a) OPN and b) a first particle comprising calcium and/or strontium for curing, alleviating and/or preventing a biofilm-related disease.

Yet an aspect of the invention pertains to the use of nanoparticle aggregates comprising a) OPN and b) a first particle comprising calcium and/or strontium, for reducing or preventing microbial biofilm growth or for removing microbial biofilm. In some embodiments of the invention, this use is not a treatment of the human or animal body by therapy. The biofilm may for example be a biofilm which is not in contact with a living human or animal.

An aspect of the invention relates to nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

Another aspect of the present invention relates to the use of nanoparticle aggregates comprising a) OPN and b) strontium phosphate, calcium phosphate and mixtures of such for reducing or preventing microbial biofilm growth.

Yet another aspect of the present invention pertains to a coating composition comprising nanoparticle aggregates comprising a) OPN and b) strontium phosphate, calcium phosphate and mixtures of such.

Still another aspect of the present invention pertains to nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5 for use as a medicament.

Yet another aspect of the present invention pertains to nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5 for use as a medicament for curing, alleviating or preventing a bacterial infection.

Yet another aspect of the present invention pertains to a dental formulation comprising nanoparticle aggregates, wherein the nanoparticle aggregates have the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

One aspect relates to a method for producing nanoparticle aggregates formed by OPN and calcium phosphate comprising:

a) Providing a first aqueous solution comprising phosphate as PO₄³⁻; wherein the pH is within the range of 6-14;

b) Providing a second aqueous solution comprising Ca²⁺ and/or Sr²⁺; wherein the pH is within the range of 6-14;

wherein either the first, second or both aqueous solutions comprise OPN;

c) Mixing said first and second solutions, thereby forming a suspension comprising nanoparticle aggregates comprising 1) OPN, 2) strontium phosphate, calcium phosphate or mixtures of such, and 3) water soluble electrolytes;

d) Optionally, removing a substantial amount of said water soluble electrolytes from the suspension;

e) Optionally, separating said nanoparticle aggregates from the water phase.

### BRIEF DESCRIPTION OF THE FIGURES

FIG. 1A shows binding of OPN to bacteria in caries model biofilms. After growth phase, a biofilm was incubated with fluorescently labelled OPN for 45 min at 35° C. Bacteria, especially chains of streptococci can be recognized, although no bacterial stain was used. The strong affinity of OPN for bacterial surfaces targets the nanoparticle aggregates towards biofilms. Bar=10 μm. FIG. 1B shows binding of calcium phosphate nanoparticle aggregates containing OPN to in vivo grown dental biofilm. The biofilm was incubated with calcium phosphate nanoparticle aggregates containing OPN for 30 min at 35° C. and stained with C-SNARF-4. The calcium phosphate nanoparticle aggregates containing OPN cluster tightly around the bacterial biofilm. Bar=20 μm;

FIGS. 2A and 2B show that bacterial growth is not influenced by OPN. S. mitis and A. naeslundii were grown in THB and THB containing 0.9 g/l OPN. Bacterial growth was monitored by spectrophotometry. Error bars indicate standard deviations. OPN was shown not to have a bactericidal or bacteriostatic effect;

FIG. 3 shows the effect of different agents on biofilm formation in the caries model, measured by crystal violet staining. Calcium phosphate nanoparticle aggregates containing OPN (HAP-OPN) strongly reduce the amount of biofilm formed in the flow cells, as compared to 1000 nm polystyrene particles, silica particles (150 nm, 500 nm and 2000 nm), OPN-free calcium phosphate particle aggregates and 0.9 g/l OPN. Error bars indicate standard deviations;

FIG. 4 shows that calcium phosphate nanoparticle aggregates containing OPN (HAP-OPN) bind significantly more crystal violet than silica particles, polystyrene particles, OPN in solution and OPN-free calcium phosphate nanoparticle aggregates (HAP). Crystal violet quantification of the amount of biofilm formed in the presence of nanoparticle aggregates containing OPN (shown in FIG. 3) overestimates the actual amount of biofilm formed in the flow cells. Error bars indicate standard deviations;

FIGS. 5A and 5B show that calcium phosphate nanoparticle aggregates containing OPN reduce the amount of biofilm grown in a caries biofilm flow cell model. Biofilms were stained with C-SNARF-4 and imaged with a confocal microscope. FIG. 5A: Biofilm grown without nanoparticle aggregates. FIG. 5B: Biofilm exposed to nanoparticle aggregates during growth. Bars=20 μm;

FIGS. 6A and 6B show that calcium phosphate nanoparticle aggregates containing OPN strongly reduce oral biofilm growth in vivo. FIG. 6A: Biofilm grown on a glass slab kept intraorally for 72 h. per day, 5-6 NaCl dips (30-60 minutes) were performed. FIG. 6B: Biofilm grown on a glass slab kept intraorally for 72 h. per day, 5-6 dips (30-60 min) with calcium phosphate nanoparticle aggregates containing OPN were performed. Both glass slabs were worn by the same study subject at the same time. Bars=20 μm;

FIGS. 7A-7E show that calcium phosphate nanoparticle aggregates containing OPN buffer the acid produced by the strains of the caries model when grown in planktonic culture;

FIGS. 8A-8E show that calcium phosphate nanoparticle aggregates containing OPN buffer the acid produced by biofilms in the five-species caries model. Biofilms were only incubated with nanoparticle aggregates after growth on THB containing glucose was finished. In biofilms that were exposed to nanoparticle aggregates, pH never dropped under 5.5, the critical value for enamel dissolution;

FIG. 9 shows X-ray diffraction patterns recorded using CuKα radiation. The diffraction patterns for the individual materials have been shifted vertically for clarity. Nanocrystalline apatite materials are obtained for an amount OPN added of 15 mg/ml. Above this concentration large amounts of amorphous material is observed; at 30 and 34 mg/ml very small diffraction peaks corresponding to nanocrystalline apatite are observed on top of the large amorphous background scattering;

FIGS. 10A and 10B show average crystallite sizes extracted from Rietveld refinement of the X-ray diffraction data in FIG. 9. The shape of nanocrystals was found to be approximately needle shaped with the long morphological axis coinciding with the crystallographic c-axis of apatite. The top panel shows results for all data while the bottom panel displays the large effect on crystallite size observed at very low concentration; the increase in crystallite size observed for 12.5 and 15 mg/ml OPN is presumably due to the formation of a mixed nanocrystalline/amorphous material;

FIG. 11 shows thermogravimetric analysis (TGA) data of nanoparticle aggregates as a function of amount of OPN added. Data have been shifted along the ordinate axis for clarity. The mass loss from 25-200° C. is assigned to loss of water, while the loss from 200 to 550° C. corresponds to organic material and the loss from 550 to 1200° C. is assigned to loss of carbonate;

FIG. 12 shows the mass fractions of water, organic and carbonate extracted from the TGA data in FIG. 11. The organic and carbonate masses have been normalized to dry material mass (the residual mass at 200° C.);

FIG. 13 shows FTIR data of nanoparticle aggregates as a function of amount of OPN added. Data have been shifted along the ordinate axis for clarity. With increasing OPN concentrations, the intensity of the amide peaks around 1300, 1550 and 1650 cm⁻¹ increases, indicating more protein is associated with the particles in the high concentration synthesis. Specific peaks for phosphate (900-1200 cm⁻¹), carbonate (840-890 cm⁻¹) and amide (1595-1720 cm⁻¹) are observed; and

FIG. 14 shows estimates of organic and carbonate content from IR data obtained as the ratios of peak areas for phosphate (900-1200 cm⁻¹), carbonate (840-890 cm⁻¹) and amide (1595-1720 cm⁻¹). Note the good agreement with the results obtained by TGA in FIG. 12.

The present invention will now be described in more detail in the following.

### DETAILED DESCRIPTION OF THE INVENTION

As stated above, an aspect of the invention pertains to nanoparticle aggregates comprising a) OPN and b) a first particle comprising calcium and/or strontium, for use as a medicament.

Thus, each of the nanoparticle aggregates preferably contains both OPN and a first particle comprising calcium and/or strontium.

Calcium is preferably present in its +2 oxidation state (Ca²⁺). Likewise, strontium is preferably used in its +2 oxidation state (Sr²⁺).

In the context of the present invention, the phrase “Y and/or X” means “Y” or “X” or “Y and X”. Along the same line of logic, the phrase “n₁, n₂, . . . , nᵢ₋₁, and/or nᵢ” means “n₁” or “n₂” or . . . or “nᵢ₋₁” or “nᵢ” or any combination of the components n₁, n₂, . . . nᵢ₋₁, and nᵢ.

As used herein the term “osteopontin” or “OPN” means osteopontin obtained from milk, including naturally occurring fragments or peptides derived from OPN by proteolytic cleavage in the milk, or genesplice-, phosphorylation-, or glycosylation variants as obtainable from the method proposed in WO 01/49741. The milk can be milk from any milk producing animals, such as cows, humans, camels, goats, sheep, dromedaries and llamas. However, OPN from bovine milk is presently preferred due to the availability. Full length osteopontin (fOPN) is an acidic, highly phosphorylated, sialic acid rich, calcium binding protein. fOPN binds 28 moles of phosphate and about 50 moles of Ca per mole. The isoelectric point of fOPN is about 3.0. The protein exists in many tissues in the body and plays a role as a signaling and regulating protein. It is an active protein in biomineralization processes. OPN is expressed by a number of cell types including bone cells, smooth muscle cells and epithelial cells.

All amounts are based on native bovine milk OPN, but can easily be corrected to the corresponding amounts of an active fraction thereof or OPN from another source. OPN or derivatives thereof can also be prepared recombinantly.

OPN is present in bovine milk, both in the form of full length bovine OPN (e.g. position 17-278 of Swiss-Prot Accession No P31096, or a peptide having at least 95% sequence identity with position 17-278 of Swiss-Prot Accession No P31096) and in the form of a long N-terminal fragment of full length bovine OPN (e.g. position 17-163 of Swiss-Prot Accession No P31096, or a peptide having at least 95% sequence identity with position 17-163 of Swiss-Prot Accession No P31096), see e.g. Bissonnette et al., Journal of Dairy Science Vol. 95 No. 2, 2012.

In the context of the present invention, the term “sequence identity” relates to a quantitative measure of the degree of identity between two amino acid sequences or between two nucleic acid sequences, preferably of equal length. If the two sequences to be compared are not of equal length, they must be aligned to the best possible fit. The sequence identity can be calculated as

(N_{ref}−N_{dif})*100)/(N_{ref}),

wherein N_{dif} is the total number of non-identical residues in the two sequences when aligned, and wherein N_{ref} is the number of residues of the reference sequences. Hence, the DNA sequence AGTCAGTC will have a sequence identity of 75% with the sequence AATCAATC (N_{dif}=2 and N_{ref}=8). A gap is counted as non-identity of the specific residue(s), i.e. the DNA sequence AGTGTC will have a sequence identity of 75% with the DNA sequence AGTCAGTC (Ndif=2 and Nref=8). Sequence identity can for example be calculated using appropriate BLAST-programs, such as the BLASTp-algorithm provided by National Center for Biotechnology Information (NCBI), USA.

For example, the OPN used in the present invention may be substantially pure full length OPN, it may be a substantially pure fragment of full length OPN and it may be a mixture comprising full length OPN and one or more fragments of OPN.

The OPN used in the present invention may be substantially pure full length bovine OPN, it may be a substantially pure, long N-terminal fragment of full length bovine OPN, and it may be a mixture comprising full length bovine OPN and the long N-terminal fragment of full length bovine OPN. Such a mixture may for example contain full length bovine OPN in an amount of 5-40% (w/w) relative to the total amount of OPN and the long n-terminal fragment of full length bovine OPN in an amount of 60-95% (w/w) relative to the total amount of OPN.

Bovine OPN is typically available in a concentration of 20 mg OPN per litre bovine milk.

Bovine OPN can be isolated by anion exchange chromatography from e. g. acid whey at pH 4.5 as described by the patent applications WO 01/497741 A2, WO 02/28413, WO 2012/117,119 or WO 2012/117,120. An OPN purity of up to 90-95% can be obtained.

The nanoparticle aggregates may furthermore contain other calcium binding peptides in addition to OPN.

The nanoparticle aggregates may, in addition to OPN, contain one or more phosphopeptides selected from the group consisting of fetuin A (FETUA) (Swiss-Prot Accession No P02765), proline-rich basic phosphoprotein 4 (PRB4) (Swiss-Prot Accession No P1 0163), matrix Gla protein (MGP) (Swiss-Prot Accession No P08493), secreted phosphoprotein 24 (SPP-24) (Swiss-Prot Accession No Q13103), Riboflavin Binding Protein (Swiss-Prot Accession No P02752), integrin binding sialophosphoprotein II (IBSP-II) (Swiss-Prot Accession No P21815), matrix extracellular bone phosphoglycoprotein (MEPE) (Swiss-Prot Accession No Q9NQ76), dentin matrix acidic phosphoprotein 1 (OMP1) (Swiss-Prot Accession No Q13316), human beta-casein, bovine beta-casein, and isoforms or phosphopeptide fragments thereof.

Another aspect of the invention pertains to nanoparticle aggregates comprising a) OPN and b) a first particle comprising calcium and/or strontium for curing, for alleviating and/or preventing a biofilm-related disease.

The nanoparticle aggregates may be used for treating human subjects or animal subjects.

In the context of the present invention the term “biofilm-related disease” pertains to a disease which is at least partly caused by biofilm contacting the human or animal body. A biofilm-related disease may for example involve a bacterial infection.

In some preferred embodiments of the invention, the biofilm-related disease is an oral disease.

The biofilm-related disease may e.g. be dental caries, gingivitis, and/or periodontitis.

The biofilm-related disease may be gingivitis. Alternatively, the biofilm-related disease is periodontitis. The biofilm-related disease may also be dental caries.

In some embodiments of the invention, the biofilm-related disease is a disease selected from the group consisting of bacterial endocarditis, chronic wound infections, implant infections, otitis media, and cystic fibrosis, and a combination thereof.

The nanoparticle aggregates may e.g. be for curing, alleviating and/or preventing a bacterial infection, e.g. a bacterial wound infection.

An aspect of the invention may for example pertain to the nanoparticle aggregates for curing, alleviating and/or preventing a bacterial infection.

The bacterial infection may for example be an oral bacterial infection, such as gingivitis. Thus, the nanoparticle aggregates may be for curing, alleviating and/or preventing gingivitis.

The nanoparticle aggregates may be for reducing or preventing microbial biofilm growth.

For example, the nanoparticle aggregates may be for reducing or preventing the formation of dental plaque.

In addition, or alternative, to reducing or preventing microbial biofilm growth the nanoparticle aggregates may be for removing microbial biofilm, such as e.g. dental plaque.

One aspect of the present invention relates to the use of nanoparticle aggregates comprising OPN and calcium phosphate for reducing or preventing microbial biofilm growth.

A biofilm is a community of microorganisms in which cells adhere to each other on a surface. These adherent cells are frequently embedded in a self-produced matrix of extracellular polymeric substance.

Another aspect of the present invention relates to the use of nanoparticle aggregates comprising OPN and strontium/calcium phosphate for reducing or preventing microbial biofilm growth.

Still another aspect relates to the use of nanoparticle aggregates comprising OPN and strontium phosphate for reducing or preventing microbial biofilm growth.

Yet another aspect relates to the use of nanoparticle aggregates comprising OPN and mixtures of strontium phosphate and calcium phosphate for reducing or preventing microbial biofilm growth.

Another aspect relates to the use of nanoparticle aggregates comprising a) OPN and b) strontium phosphate, calcium phosphate and mixtures of such, for reducing or preventing microbial biofilm growth.

In the present context, nanoparticle aggregates are taken to mean collections of nanoparticles, wherein said nanoparticles do not readily separate from each other upon mechanical stimulus such as stirring or low-power ultrasonication. As an example, OPN nanoparticles and calcium phosphate nanoparticles combine to form nanoparticle aggregates.

Various sizes of nanoparticle aggregates may be used in the present invention. In some embodiments of the invention the nanoparticle aggregates have a hydrodynamic radius of at most 5 micron. For example, the nanoparticle aggregates may have a hydrodynamic radius of at most 2 micron. The nanoparticle aggregates may e.g. have a hydrodynamic radius of at most 1 micron. Alternatively, the nanoparticle aggregates may have a hydrodynamic radius of at most 0.7 micron.

Even smaller nanoparticle aggregates may be preferred. Thus, in some preferred embodiments of the invention the nanoparticle aggregates have a hydrodynamic radius of at most 0.5 micron. For example, the nanoparticle aggregates may have a hydrodynamic radius of at most 0.4 micron. The nanoparticle aggregates may e.g. have a hydrodynamic radius of at most 0.3 micron. Alternatively, the nanoparticle aggregates may have a hydrodynamic radius of at most 0.2 micron, such as at most 0.1 micron.

The hydrodynamic radius is preferably determined using Dynamic Light Scatter (DLS).

Typically, the nanoparticle aggregates have a hydrodynamic radius of at least 5 nm, and preferably at least 10 nm.

As said, the first particle comprises calcium and/or strontium.

In some preferred embodiments of the invention the first particle comprises calcium.

In some preferred embodiments of the invention the first particle comprises strontium.

In some preferred embodiments of the invention the first particle comprises calcium and strontium.

The first particle may e.g. comprise, or even consist of, a salt comprising calcium and/or strontium.

The first particle may comprises at least 50% (w/w) of the salt of calcium and/or strontium relative to the total weight of the first particle. For example, the first particle may comprise at least 70% (w/w) of the salt of calcium and/or strontium. The first particle may e.g. comprise at least 80% (w/w) of the salt of calcium and/or strontium. Alternatively, the first particle may comprise at least 90% (w/w) of the salt of calcium and/or strontium, such as at least 95% (w/w.)

The salt may be an organic salt containing calcium and/or strontium and an appropriate organic anion, e.g. in the form of an anionic organic polymer.

Alternatively, the salt may be an inorganic salt containing calcium and/or strontium and an appropriate inorganic anion. None-limiting examples of such inorganic anions are a phosphate species, a sulfate, a carbonate, or a mixture thereof. Thus, the inorganic salt may comprise a phosphate species, sulfate, and/or carbonate.

The phosphate species may for example be phosphate (PO₄³⁻), monohydrogen phosphate (HPO₄²⁻), a pyrophosphate, or diphosphate (P₂O₇⁴⁻). It is presently preferred that the phosphate species is phosphate (PO₄³⁻), or alternatively a combination of phosphate (PO₄³⁻) and monohydrogen phosphate (HPO₄²⁻).

Thus, in some preferred embodiments of the invention, the first particle comprises, or even consists of, an inorganic salt of calcium and/or strontium.

Preferably, the first particle comprises at least 50% (w/w) of inorganic salt of calcium and/or strontium relative to the total weight of the first particle. For example, the first particle may comprise at least 70% (w/w) of inorganic salt of calcium and/or strontium. The first particle may e.g. comprise at least 80% (w/w) of inorganic salt of calcium and/or strontium. Alternatively, the first particle may comprise at least 90% (w/w) of inorganic salt of calcium and/or strontium, such as at least 95% (w/w.)

In some preferred embodiments of the invention the first particle is capable of releasing calcium and/or strontium. This release of calcium and/or strontium preferably occurs when the nanoparticle aggregates contact or are near the biofilm. For example, the nanoparticle aggregates are preferably capable of releasing calcium and/or strontium when present in a liquid film of the oral cavity, such as e.g. saliva or an oral biofilm.

In some presently preferred embodiments of the invention, the first particle is a nanoparticle, i.e. it has a hydrodynamic radius of at most 1 micron. For example, the hydrodynamic radius of the first particle may be at most 0.8 micron. The hydrodynamic radius of the first particle may e.g. be at most 0.6 micron. Alternatively, the hydrodynamic radius of the first particle may be at most 0.4 micron.

Even smaller particles may be preferred, thus the first particle may have a hydrodynamic radius of at most 0.2 micron. For example, the hydrodynamic radius of the first particle may be at most 0.1 micron. The hydrodynamic radius of the first particle may e.g. be at most 0.05 micron. Alternatively, the hydrodynamic radius of the first particle may be at most 0.01 micron.

Typically, the hydrodynamic radius of the first particle is at least 3 nm and preferably at least 5 nm.

In some preferred embodiments of the invention at least some of the nanoparticle aggregates contain a single first particle to which one or more OPN molecules are bound. The first particle of the nanoparticle aggregates may for example be surrounded by a monolayer of OPN. Examples of such nanoparticle aggregates can be found in Holt et al. (FEBS Journal; vol. 276; pages 2308-2323; 2009).

In some preferred embodiments of the invention at least some of the nanoparticle aggregates comprise a second particle, and possibly even further particles, of the same type as the first particle. Such nanoparticle aggregates are therefore more complex structures each contain multiple particles containing calcium and/or strontium in addition to multiple OPN molecules.

In some preferred embodiments of the invention, the first particle comprises, or even consists essentially of, calcium phosphate. For example, the first particle may comprise a total amount of calcium phosphate of at least 50% (w/w) relative to the weight of the first particle. The first particle may e.g. comprise a total amount of calcium phosphate of at least 60% (w/w) relative to the weight of the first particle, such as at least 70% (w/w) or even at least 80% (w/w). Alternatively, the first particle may comprise a total amount of calcium phosphate of at least 90% (w/w) relative to the weight of the first particle. The remaining part of the first particle may e.g. be impurities from the sources of calcium and phosphate used to prepare the first particle.

The first particle may e.g. have the stoichiometric formula:

A′_{10-X}A_{X}(PO₄)_{6-Y}B_{Y}(OH)_{2-z}C_{z}.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, Cl, Br, I, (CO3), H₂O, vacancy and mixtures thereof, and Z=0-2; and wherein n=0-10; wherein the molar ratio between (PO₄) and (HPO₄) is above 2.5.

Another aspect of the invention relates to nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

In the present context, the term ‘vacancy’ is to be understood as a type of point defect in the mineral. Mineral crystals inherently possess imperfections, often referred to as ‘crystalline defects’. A defect wherein an atom is missing from one of the lattice sites is known as a ‘vacancy’ defect.

In one embodiment, A′ is Ca.

In another embodiment, A′ is Sr.

In yet another embodiment, A′ is a mixture of Ca and Sr.

In one embodiment, A is selected from the group consisting of Na, K, Rb, Cs, Mg, Zn, Ba, vacancy and mixtures thereof.

In another embodiment, A is selected from the group consisting of Na, K, Mg, Zn, Ba, vacancy and mixtures thereof.

In still another embodiment, A is selected from the group consisting of Na, K, vacancy and mixtures thereof.

In yet another embodiment, B is selected from the group consisting of (CO₃), (HPO₄), (H₂PO₄), H₂O, vacancy and mixtures thereof.

In yet another embodiment, C is selected from the group consisting of F, CI, vacancy and mixtures thereof.

In one embodiment, the molar ratio between (PO₄) and (HPO₄) is above 2.5, such as above 5, such as above 10, such as above 15, such as above 20, such as above 25, such as above 30, such as above 35, such as above 40, such as above 45, such as above 50, such as above 100, such as above 200, such as above 300, such as above 400, such as above 500, such as above 1000, such as above 10,000, such as above 20,000, such as above 50,000, such as above 100,000.

In yet another embodiment, A′ is a mixture of Ca and Sr; wherein the ratio between Ca and Sr is within the range of 1:1000 to 1000:1, such as within the range of 1:900 to 900:1, e.g. 1:850 or 850:1, such as within the range of 1:800 to 800:1, e.g. 1:750 or 750:1, such as within the range of 1:700 to 700:1, e.g. 1:650 or 650:1, such as within the range of 1:600 to 600:1, e.g. 1:550 or 550:1, such as within the range of 1:500 to 500:1, e.g. 1:450 or 450:1, such as within the range of 1:400 to 400:1, e.g. 1:350 or 350:1, such as within the range of 1:300 to 300:1, e.g. 1:250 or 250:1, such as within the range of 1:200 to 200:1, e.g. 1:150 or 150:1, such as within the range of 1:100 to 100:1, e.g. 1:75 or 75:1, such as within the range of 1:50 to 50:1, e.g. 1:25 or 25:1, such as within the range of 1:15 to 15:1, e.g. 1:10 or 10:1, such as within the range of 1:5 to 5:1, e.g. 1:5 or 5:1, such as within the range of 1:2.5 to 2.5:1, e.g. 1:2.5 or 2.5:1, e.g. 1:1.

In one embodiment, X is within the range of 0-9, such as within the range of 0-8, such as within the range of 0-7, such as within the range of 0-6, such as within the range of 0-5, such as within the range of 0-4, such as within the range of 0-3, such as within the range of 0-2, such as within the range of 0-1.

In another embodiment, X is within the range of 0-9.5, such as within the range of 0.1-9.0, e.g. 0.2, such as within the range of 0.3-8.5, e.g. 0.4, such as within the range of 0.5-8.0, e.g. 0.6, such as within the range of 0.7-7.5, e.g. 0.8, such as within the range of 0.9-7.0, e.g. 1.0, such as within the range of 1.1-6.5, e.g. 1.2, such as within the range of 1.3-6.0, e.g. 1.4, such as within the range of 1.5-5.5, e.g. 1.6, such as within the range of 1.7-5.0, e.g. 1.8, such as within the range of 1.9-4.5, e.g. 2.0, such as within the range of 2.1-4.0, e.g. 2.2, such as within the range of 2.3-3.5, e.g. 2.4, such as within the range of 2.5-3.5, e.g. 3.0.

In one embodiment, Y is within the range of 0-5, such as within the range of 0-4, such as within the range of 0-3, such as within the range of 0-2, such as within the range of 0-1.

In another embodiment, Y is within the range of 0-4.5, such as within the range of 0.1-4.0, e.g. 0.2, such as within the range of 0.3-4.0, e.g. 0.4, such as within the range of 0.4-4.0, e.g. 0.5, such as within the range of 0.6-4.0, e.g. 0.7, such as within the range of 0.8-4.0, e.g. 0.9, such as within the range of 1.0-3.5, e.g. 1.2, such as within the range of 1.3-3.5, e.g. 1.4, such as within the range of 1.5-3.5, e.g. 1.6, such as within the range of 1.7-3.5, e.g. 1.8, such as within the range of 1.9-3.5, e.g. 2.0, such as within the range of 2.1-3.0, e.g. 2.2, such as within the range of 2.3-3.0, e.g. 2.4, such as within the range of 2.5-3.0.

In one embodiment, Z is within the range of 0-2, such as within the range of 0-1.

In another embodiment, Z is within the range of 0-1.9, such as within the range of 0.1-1.9, e.g. 0.2, such as within the range of 0.3-1.9, e.g. 0.4, such as within the range of 0.5-1.9, e.g. 0.6, such as within the range of 0.7-1.9, e.g. 0.8, such as within the range of 0.9-1.9, e.g. 1.0, such as within the range of 1.0-1.8, e.g. 1.1, such as within the range of 1.2-1.7, e.g. 1.3, such as within the range of 1.4-1.6, e.g. 1.5.

In one embodiment, B is selected from the group consisting of (CO3), H₂O, vacancy and mixtures thereof.

In another embodiment, A is selected from the group consisting of Na, H₂O, vacancy and mixtures thereof.

In yet another embodiment, B is selected from the group consisting of (CO3), H₂O, vacancy and mixtures thereof; and A is selected from the group consisting of Na, H₂O, vacancy and mixtures thereof.

In yet another embodiment, B is selected from the group consisting of (CO3), H₂O, vacancy and mixtures thereof; and A is selected from the group consisting of K, H₂O, vacancy and mixtures thereof.

In one embodiment, m is within the range of 1*10⁻¹⁰ to 0.25, such as within the range of 1*10⁻¹⁰ to 0.20, e.g. within the range of 1*10⁻¹⁰ to 0.15, such as within the range of 1*10⁻¹⁰ to 0.10, e.g. within the range of 1*10⁻¹⁰ to 0.09, such as within the range of 1*10⁻¹⁰ to 0.08, e.g. within the range of 1*10⁻¹⁰ to 0.07, such as within the range of 1*10⁻¹⁰ to 0.06, e.g. within the range of 1*10⁻¹⁰ to 0.05, such as within the range of 1*10⁻¹⁰ to 0.04, e.g. within the range of 1*10⁻¹⁰ to 0.03, such as within the range of 1*10⁻¹⁰ to 0.02, e.g. within the range of 1*10⁻¹⁰ to 0.01.

In yet another embodiment, m is within the range of 1*10⁻¹⁰ to 0.30, such as within the range of 1*10⁻⁹ to 0.20, e.g. within the range of 1*10⁻⁸ to 0.15, such as within the range of 1*10⁻⁷ to 0.10, e.g. within the range of 1*10⁻⁶ to 0.09, such as within the range of 1*10⁻⁵ to 0.08, e.g. within the range of 1*10⁻⁴ to 0.07, such as within the range of 0.001 to 0.06, e.g. within the range of 0.002 to 0.05, such as within the range of 0.003 to 0.04, e.g. within the range of 0.005 to 0.03, such as within the range of 0.006 to 0.02, e.g. within the range of 0.007 to 0.01.

In still another embodiment, m is within the range of 0.00016 to 0.011, such as 0.00016 to 0.009, such as 0.0009 to 0.0035, such as 0.0018 to 0.003.

In another embodiment, n is within the range of 0-100, such as 0.01-100, e.g. 0.05, such as within the range of 0.1-90, e.g. 0.2, such as within the range of 0.3-85, e.g. 0.4, such as within the range of 0.5-80, e.g. 0.6, such as within the range of 0.7-75, e.g. 0.8, such as within the range of 0.9-70, e.g. 1.0, such as within the range of 1.2-65, e.g. 1.4, such as within the range of 1.6-60, e.g. 1.8, such as within the range of 2.0-55, e.g. 2.2, such as within the range of 2.4-50, e.g. 2.4, such as within the range of 2.6-45, e.g. 2.8, such as within the range of 3.0-40, e.g. 3.2, such as within the range of 3.4-35, e.g. 3.6, such as within the range of 3.8-30, e.g. 4.0, such as within the range of 4.2-25, e.g. 4.4, such as within the range of 4.6-20, e.g. 4.8, such as within the range of 5.0-15, e.g. 5.2, such as within the range of 5.4-14, e.g. 5.6, such as within the range of 5.8-13, e.g. 6.0, such as within the range of 6.2-12, e.g. 6.4, such as within the range of 6.6-11, e.g. 6.8, such as within the range of 7.0-10, e.g. 7.2, such as within the range of 7.4-9, e.g. 7.6, such as within the range of 7.8-8.8, e.g. 8.0.

Still another aspect of the present invention pertains to nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5 for use as a medicament.

Yet another aspect of the present invention pertains to nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5 for use as a medicament for curing, alleviating or preventing a bacterial infection.

In one embodiment, A is selected from the group consisting of Zn, vacancy and mixtures thereof.

In another embodiment, B is selected from the group consisting of (CO3), H₂O, vacancy and mixtures thereof.

In yet another embodiment, C is selected from the group consisting of F, H₂O, vacancy and mixtures thereof.

In yet another embodiment, A is selected from the group consisting of Na, H₂O, vacancy and mixtures thereof, and X=0-9; wherein B is (CO₃) and Y=0-5; wherein C is selected from the group consisting of H₂O, vacancy and mixtures thereof, and Z=0-2; wherein n=0-10 and m>0.

In one embodiment, A is selected from the group consisting of Na, H₂O, vacancy and mixtures thereof, and X=0-2; wherein B is (CO₃) and Y=0-1; wherein C is selected from the group consisting of H₂O, vacancy and mixtures thereof, and Z=0-2; wherein n=0-10 and m>0.

In one embodiment, A is selected from the group consisting of Na, Zn, H₂O, vacancy and mixtures thereof, and X=0-9; wherein B is selected from the group consisting of (CO₃), (HPO₄), (H₂PO₄), H₂O, vacancy and mixtures thereof, and Y=0-5; wherein C is selected from the group consisting of F, (CO3), H₂O, vacancy and mixtures thereof, and Z=0-2; wherein n=0-10 and m>0; wherein the molar ratio between (PO₄) and (HPO₄) is above 2.5.

In another embodiment, the nanoparticle aggregates are amorphous. The first particle may for example be substantially amorphous.

Alternatively, the first particle may be substantially crystalline.

In yet another embodiment, the nanoparticle aggregates contain crystalline material matching the X-ray diffraction pattern of hydroxylapatite.

In the present context, the term ‘nanocrystalline’ is to be understood as a crystalline material where at least one dimension of the nanocrystals is smaller than 100 nm.

In still another embodiment, the nanoparticle aggregates contain crystalline material matching the X-ray diffraction pattern of hydroxylapatite and said crystalline material is nanocrystalline.

In one embodiment, the nanoparticle aggregates contain crystalline material matching the X-ray diffraction pattern of hydroxylapatite, and said crystalline material is nanocrystalline; wherein said nanocrystals have anisotropic crystallite size with the crystallographic c-axis coinciding with the largest morphological axis of the crystallites.

In another embodiment, the nanoparticle aggregates contain crystalline material matching the X-ray diffraction pattern of hydroxylapatite and said crystalline material is nanocrystalline in the sense that at least one dimension of the nanocrystals is smaller than 90 nm, such as smaller than 80 nm, e.g. smaller than 70 nm, such as smaller than 60 nm, e.g. smaller than 50 nm, such as smaller than 40 nm, e.g. smaller than 30 nm, such as smaller than 20 nm, e.g. smaller than 10 nm.

The inventors have seen indications that the present nanoparticle aggregates are particularly suitable for preventing or destabilising biofilm which contains one or more species of OPN-binding bacteria.

In some embodiments of the invention the biofilm contains bacteria having an OPN binding capacity of at least 50 OPN molecules per cell. For example, the biofilm may contain bacteria having an OPN binding capacity of at least 100 OPN molecules per cell. The biofilm may e.g. contain bacteria having an OPN binding capacity of at least 200 OPN molecules per cell. Alternatively, the biofilm may contain bacteria having an OPN binding capacity of at least 400 OPN molecules per cell.

The OPN binding capacity of a bacterial strain is measured according to Rydén et al., Eur. J. Biochem., 184, 331-336 (1989) using full length OPN isolated from bovine milk.

In some preferred embodiments of the invention the biofilm contains bacteria having an OPN binding capacity of at least 800 OPN molecules per cell. For example, the biofilm may contain bacteria having an OPN binding capacity of at least 2,000 OPN molecules per cell. The biofilm may e.g. contain bacteria having an OPN binding capacity of at least 10,000 OPN molecules per cell. Alternatively, the biofilm may contain bacteria having an OPN binding capacity of at least 50,000 OPN molecules per cell, such as e.g. at least 100,000 OPN molecules per cell or even at least 500,000 OPN molecules per cell.

The biofilm may e.g. contain bacteria having an OPN binding capacity of at least 1,000,000 OPN molecules per cell.

For example, the biofilm may contain, or even consist of, one or more bacteria selected from the group consisting of Streptococcus spp., Staphylococcus spp., Pseudomonas spp. Actinomyces spp., Lactobacillus spp., Aggregatibacter spp., Bacteroides spp., Listeria spp., Campylobacter spp., Eikenella spp., Porphyromonas spp., Prevotella spp., Treponema spp., and combinations thereof.

In the case of gingivitis, the biofilm typically contains one or more of the bacteria Aggregatibacter actinomycetemcomitans, Bacteroides forsythus, Campylobacter rectus, Eikenella corrodens, Porphyromonas gingivalis, Prevotella intermedia, Prevotella nigrescens, and/or Treponema denticola.

In the case of dental caries the biofilm typically contains one or more of the bacteria Streptococcus oralis, Streptococcus downei, Streptococcus mitis, Streptococcus sanguinis and Actinomyces naeslundii.

The biofilm may furthermore, in addition to the OPN-binding bacteria, contain bacteria having no or low binding to OPN.

One aspect relates to the use of nanoparticle aggregates comprising OPN and calcium phosphate for reducing or preventing bacterial biofilms formed by Streptococcus spp. and/or Actinomyces spp.

Another aspect of the present invention relates to the use of nanoparticle aggregates comprising OPN and strontium/calcium phosphate for reducing or preventing bacterial biofilms formed by Streptococcus spp. and/or Actinomyces spp.

Still another aspect relates to the use of nanoparticle aggregates comprising OPN and strontium phosphate for reducing or preventing bacterial biofilms formed by Streptococcus spp. and/or Actinomyces spp.

Yet another aspect relates to the use of nanoparticle aggregates comprising OPN and mixtures of strontium phosphate and calcium phosphate for reducing or preventing bacterial biofilms formed by Streptococcus spp. and/or Actinomyces spp.

Another aspect relates to the use of nanoparticle aggregates comprising a) OPN and b) strontium phosphate, calcium phosphate and mixtures of such, for reducing or preventing bacterial biofilms formed by Streptococcus spp. and/or Actinomyces spp.

Yet another aspect relates to the use of nanoparticle aggregates comprising OPN and calcium phosphate for reducing or preventing bacterial adhesion of Streptococcus spp. and/or Actinomyces spp.

Many problems occur in connection with care of the teeth, cosmetically as well as therapeutically, such as formation of dental biofilm (plaque), staining of teeth due to bacterial products, formation of dental calculus (tartar) dental caries, root canal infections and periodontal disease.

Dental plaque is a complex biofilm that accumulates on the hard tissues (teeth) in the oral cavity. Although dental biofilms harbour over 500 bacterial species, colonization follows a regimented pattern with adhesion of initial colonizers to the enamel salivary pellicle followed by secondary colonization through bacterial co-adhesion. It is well known that a range of Streptococcus species and Actinomyces species belong to the early colonizers. It is therefore important to control the adhesion and subsequent biofilm formation of these bacteria. A variety of adhesins and receptors are involved in bacterial adhesion to saliva-coated surfaces, in bacterial coaggregation, in bacterium-matrix interactions and contribute to biofilm development and ultimately to diseases such as caries, endodontic infections and periodontal disease.

One aspect relates to the use of nanoparticle aggregates comprising OPN and calcium phosphate for reducing or preventing oral biofilm growth.

When used to treat, prevent or reduce oral biofilm it is preferred that the nanoparticle aggregates are administered orally. It is furthermore preferred that the nanoparticle aggregates are present in a formulation suitable for oral administration.

Another aspect of the present invention relates to the use of nanoparticle aggregates comprising OPN and strontium/calcium phosphate for reducing or preventing oral biofilm growth.

Still another aspect relates to the use of nanoparticle aggregates comprising OPN and strontium phosphate for reducing or preventing oral biofilm growth.

Yet another aspect relates to the use of nanoparticle aggregates comprising OPN and mixtures of strontium phosphate and calcium phosphate for reducing or preventing oral biofilm growth.

Another aspect relates to the use of nanoparticle aggregates comprising a) OPN and b) strontium phosphate, calcium phosphate and mixtures of such, for reducing or preventing oral biofilm growth.

Still another aspect relates to the use of nanoparticle aggregates comprising a) OPN and b) strontium phosphate, calcium phosphate and mixtures of such, for reducing or preventing oral biofilm adhesion.

In one embodiment, the nanoparticle aggregates comprising OPN and calcium phosphate have the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

Another aspect of the invention pertains to a method of curing, for alleviating and/or preventing a biofilm-related disease of an animal or human subject by administering to the subject nanoparticle aggregates as defined herein.

An aspect of the present invention relates to the use nanoparticle aggregates as defined herein for reducing, removing and/or preventing bad breath.

A further aspect of the invention pertains to a method of reducing or preventing microbial biofilm growth in or on an animal or human subject by administering to the subject nanoparticle aggregates as defined herein.

As stated above, the biofilm to be treated, reduced or prevented may e.g. be an oral biofilm, in which case oral administration of the nanoparticle aggregates is preferred.

The animal subject may e.g. be a domesticated animal such as a domesticated mammal, a domesticated fish or a domesticated bird.

A further aspect of the invention pertains to a method of reducing, removing and/or preventing bad breath of animal or human subject by administering to the subject nanoparticle aggregates as defined herein. The nanoparticle aggregates are preferably administered orally.

Yet an aspect of the invention pertains to the use of nanoparticle aggregates comprising a) OPN and b) a first particle comprising calcium and/or strontium, for reducing or preventing microbial biofilm growth. In some embodiments of the invention, it is required that this use is not a treatment of the human or animal body by therapy.

The biofilm can in theory be any of the biofilms mentioned herein, and may for example contain one more of the following bacteria: Streptococcus spp., Staphylococcus spp., Pseudomonas spp. Actinomyces spp., Lactobacillus spp., Aggregatibacter spp., Bacteroides spp., Listeria spp., Campylobacter spp., Eikenella spp., Porphyromonas spp., Prevotella spp., Treponema spp.

However, in some embodiments of the invention, the above use is not a treatment of the human or animal body by therapy. The biofilm may for example be a biofilm which is not in contact with a living human or a living animal.

Yet another aspect of the present invention pertains to a dental formulation comprising nanoparticle aggregates as defined herein.

The dental formulation may for example comprise the nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

The dental formulations can be any dentifrice or related product of relevance in oral hygiene, such as for example toothpowder, tooth gel, tooth varnish, dental mouthwash, mouth spray or chewing gum.

In one embodiment, the dental formulation is in the form of a toothpaste, toothpowder, tooth gel, tooth varnish, dental mouthwash, mouth spray or chewing gum.

As disclosed in WO 2005/053,628, the amount of osteopontin is normally between about 50 mg OPN and about 1500 mg osteopontin per kg dental formulation, and that smaller amounts will also have an effect. Higher amounts can be used, but the effect will not be essentially increased. A useful amount is 100-1000 mg OPN per kg, preferably 200-500 mg, and most preferred about 350 mg. Higher amounts will presumably not give better results and are therefore not recommended, because OPN is a rather expensive ingredient. Surprisingly, nanoparticle aggregates comprising OPN and calcium phosphate have been shown by the inventors of the present invention to have a synergistic effect at reducing or preventing microbial biofilm growth. Therefore, the present invention reduces the effective amount of osteopontin per kg dental formulation.

In the present context, the term “oral biofilm growth” means biofilm growth on oral hard and soft tissues (oral mucosa, tongue, tooth surfaces) and biofilm growth on materials inserted in the oral cavity (implants, orthodontic brackets, and restorative materials such as fillings, crowns and dentures).

Preferred compositions of the subject invention are as already mentioned in the form of tooth-pastes, tooth varnish, tooth-gels and tooth powders. Components of such toothpaste and tooth-gels include one or more of the following: a dental abrasive (from about 10% to about 50%), a surfactant (from about 0.5% to about 10%), a thickening agent (from about 0.04% to about 0.5%), a humectant (from about 0.1% to about 3%), a flavouring agent (from about 0.04% to about 2%), a sweetening agent (from 0.1% to about 3%), a colouring agent (from about 0.01% to about 0.5%) and water (from 2 to 45%).

Unless it is stated otherwise, the percentages of the components which form part of the compositions or products of the present invention are weight percent relative to the total weight of the composition or product.

Caries controlling agents may contain from 0.001% to about 1% nanoparticle aggregates comprising OPN and calcium phosphate. Anti-calculus agents contain from about 0.1% to about 13% nanoparticle aggregates comprising OPN and calcium phosphate.

Tooth powders, of course, are substantially free from all liquid components.

Other preferred compositions of the subject invention are dental mouth washes, including mouth sprays. Components of such mouth washes and mouth sprays typically include one or more of the following: water (from about 45% to about 95%), ethanol (from about 0% to about 25%), a humectant (from about 0% to about 50%), a surfactant (from about 0.01% to about 7%), a flavouring agent (from about 0.04% to about 2%), a sweetening agent from (from about 0.1% to about 3%), and a colouring agent (from about 0.001% to about 0.5% anti-caries agent including nanoparticle aggregates comprising OPN and calcium phosphate, from about 0.001% to 1% and an anti-calculus agent (from about 0.1% to about 13%).

A third area of application is in chewing gum formulations of various compositions in general terms.

Strontium (Sr) has been reported to promote bone formation and is approved for the treatment of osteoporosis.

Still another aspect relates to a coating composition comprising nanoparticle aggregates as defined herein.

The coating composition may for example contain nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

The coating composition may e.g. comprise nanoparticle aggregates comprising OPN and calcium phosphate.

The coating composition may be used in connection with bone disease, bone fracture or implants.

One aspect relates to the use of the coating compositions according to the present invention to coat medical devices.

Another aspect relates to a food or beverage product comprising nanoparticle aggregates as defined herein.

The food or beverage product may for example comprise nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

An object of the present invention is to improve the reduction of biofilm on surfaces.

One aspect relates to a product comprising a bulk part and a surface region; wherein a first surface region coating is coated on at least a first part of said surface region; said first surface region coating comprising nanoparticle aggregates as defined herein.

The first surface region coating may e.g. comprise nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

In one embodiment, the surface region comprises a material selected from the group consisting of metals, metal oxides, inorganic materials, organic materials, and polymers.

In another embodiment, the surface region is positively charged.

In yet another embodiment, the surface region is negatively charged.

In still another embodiment, the surface region is uncharged.

In the present context, the term ‘anti-biofilm agent’ is to be understood as an agent that prevents and/or reduces microbial adhesion to surfaces and/or prevents and/or reduces microbial biofilm formation and/or disrupts and/or destabilizes microbial biofilms.

One aspect of the invention relates to an anti-biofilm agent comprising a) OPN and b) a first particle comprising calcium and/or strontium.

The anti-biofilm agent may for example comprise nanoparticle aggregates comprising a) OPN and b) strontium phosphate, calcium phosphate or mixtures of such.

The anti-biofilm agent may e.g. comprise nanoparticle aggregates having the stoichiometric formula

A′_{10-X}A_{X}(PO₄)_{6-Y}B_{Y}(OH)_{2-z}C_{z}.OPNₘ.(H₂O)ₙ; wherein A′ is selected from the group consisting of Ca, Sr and mixtures thereof; wherein A is selected from the group consisting of Li, Na, K, Rb, Cs, Be, Mg, Zn, Ba, H₂O, vacancy and mixtures thereof, and X=0-9; wherein B is selected from the group consisting of (CO₃), (SO₄), (HSO₄), (HPO₄), (H₂PO₄), H₂O, vacancy and mixtures thereof, and Y=0-5; wherein C is selected from the group consisting of F, CI, Br, I, (CO3), H₂O, vacancy and mixtures thereof, and Z=0-2; wherein n=0-10 and m>0; wherein the molar ratio between (PO₄) and (HPO₄) is above 2.5.

Another aspect of the invention relates to a method for producing nanoparticle aggregates formed by OPN and calcium phosphate comprising:

a) Providing a first aqueous solution comprising phosphate as PO₄³⁻; wherein the pH is within the range of 6-14;

b) Providing a second aqueous solution comprising Ca²⁺ and/or Sr²⁺; wherein the pH is within the range of 6-14;

wherein either the first, second or both aqueous solutions comprises OPN;

c) Mixing said first and second solutions, thereby forming a suspension comprising nanoparticle aggregates comprising 1) OPN, 2) strontium phosphate, calcium phosphate or mixtures of such, and 3) water soluble electrolytes;

d) Optionally, removing a substantial amount of said water soluble electrolytes from the suspension;

e) Optionally, separating said nanoparticle aggregates from the water phase.

In the present context, the term ‘aqueous solution’ is to be understood as a liquid matter comprising at least 50% w/w of water.

In the present context, the term ‘suspension’ is to be understood as a heterogeneous fluid containing solid particles that are sufficiently large for sedimentation.

In the present context, the term ‘electrolyte’ is to be understood as a substance containing free ions that make the substance electrically conductive. In the present invention, the electrolyte must be soluble in water.

In one embodiment, the pH of the water phase of the suspension is above 7, such as within the range of 7.4-14.0, e.g. above 7.6, such as within the range of 7.8-13.5, e.g. above 8.0, such as within the range of 8.5-13.0, e.g. above 9.0, such as within the range of 9.5-12.5, e.g. above 10.0, such as within the range of 10.5-12.0, e.g. above 11.0.

In one embodiment, the process further comprises the step d).

In another embodiment, the process further comprises the step d), wherein the separation is performed by dialysis.

In the present context, the term ‘dialysis’, is to be understood as separation of suspended colloidal particles from dissolved ions or molecules of small dimensions (crystalloids) by means of their unequal rates of diffusion through the pores of semipermeable membranes.

In one embodiment, the process further comprises the step e).

In another embodiment, the total concentration of osteopontin in the first and second aqueous solutions is above 2.5 mg/ml, such as within the range of 3-1000 mg/ml, e.g. above 5 mg/ml, such as within the range of 10-500 mg/ml, e.g. above 12 mg/ml, such as within the range of 15-100 mg/ml, e.g. above 18 mg/ml, such as within the range of 20-50 mg/ml, e.g. above 25 mg/ml, such as within the range of 30-45 mg/ml, e.g. above 35 mg/ml.

In one embodiment, the process further comprises the step f) sterilizing the obtained nanoparticle aggregates.

In another embodiment, the process further comprises the step f), wherein the sterilization step is performed by heating.

In yet another embodiment, the process further comprises the step f), wherein the sterilization step is performed by heating to 80° C. and keeping the temperature at this level for 1 hour or more.

The inventors have found that there is an upper limit of the osteopontin concentration of about 33 wt % of dried mass (i.e. referring to the mass obtained by drying at 200° C.), where no more osteopontin seems to be included in the nanoparticle aggregates. This sets in at an added OPN amount of about 20 mg/I. Hence, in one embodiment, the total concentration of osteopontin in the first and second aqueous solutions is within the range of 2.5-35 mg/ml. However, this limit may change depending on the concentration of different water soluble electrolytes.

In one embodiment, the osteopontin is only present in the first aqueous solution.

In another embodiment, the osteopontin is only present in the second aqueous solution.

In yet another embodiment, the osteopontin is present in both the first and the second aqueous solution.

In one embodiment, step c) is performed at a temperature within the range of 5-80 degrees Celsius, such as within the range of 10-50° C., e.g. within the range of 20-40° C., such as within the range of 22-28° C.

In one embodiment, either the first, second or both aqueous solutions comprises a Ca/Sr-binding fluorescent dye. An additional Ca/Sr-binding dye in the solutions will result in fluorescent (if fluorescent dye) or colored nanoparticle aggregates.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Synthesis of Nanoparticle Aggregates

For synthesis of nanoparticle aggregates, the following method was used
1) Three aqueous solutions were prepared in equal volumes, one containing 0.36 M Na₃PO₄ (conveniently made by making a solution that was 0.36 M NaH₂PO₄ and 0.72 M NaOH), the second containing 0.6 M CaCl₂ and the third containing three times the desired final amount of OPN.
2) The CaCl₂ and OPN solutions were mixed first, and then the Na₃PO₄ solution was added. This mixing produced a turbid gel-like suspension. The gel-like structure broke down under stirring on a magnet stirrer. The solution was stirred for 24 h at 25° C. using a custom designed temperature controlled water bath.
3) After 24 h, the solution was transferred to dialysis bags to remove excess NaCl. The dialysis was carried out for 24 h in a reservoir containing 100 times the solution volume of water that was slowly stirred by a magnetic stirrer. The reservoir water was changed after 12 h of dialysis.
4) After dialysis the nanoparticle aggregates were centrifuged at 4800 rpm and washed with water two times.
5) After washing, the nanoparticle aggregates were dried at 60° C. before analysis.

The results discussed below were obtained by reaction (step 2 above) at 25° C.; the reaction can equally well be performed at other temperatures both higher and lower. At increased temperatures the reaction is faster. The synthesis has also been executed using K₃PO₄ as the phosphate source. The reaction can also be completed with lower (higher) concentrations of reagents with ensuing smaller (higher) yields.

Samples with different amounts of OPN were produced. Samples containing 0, 0.001, 0.0025, 0.01, 0.025, 0.1, 0.25, 1.0, 2.5, 5.0, 10.0, 12.5, 15.0, 20.0, 25.0, 30.0 and 34.0 mg/mL were synthesised (17 different concentrations in total).

Nanoparticle aggregates for biofilm experiments were synthesized with an OPN amount added of 12.5 mg/ml as described above with the modification that the dialysis reservoir liquid had pH and 0.9 wt % NaCl. After 24 h dialysis, the resulting suspension was sterilized by heating the suspension in a closed container at 80° C. for 1 h. The above steps 4) and 5) were omitted in the production of the nanoparticle aggregates for biofilm experiments.

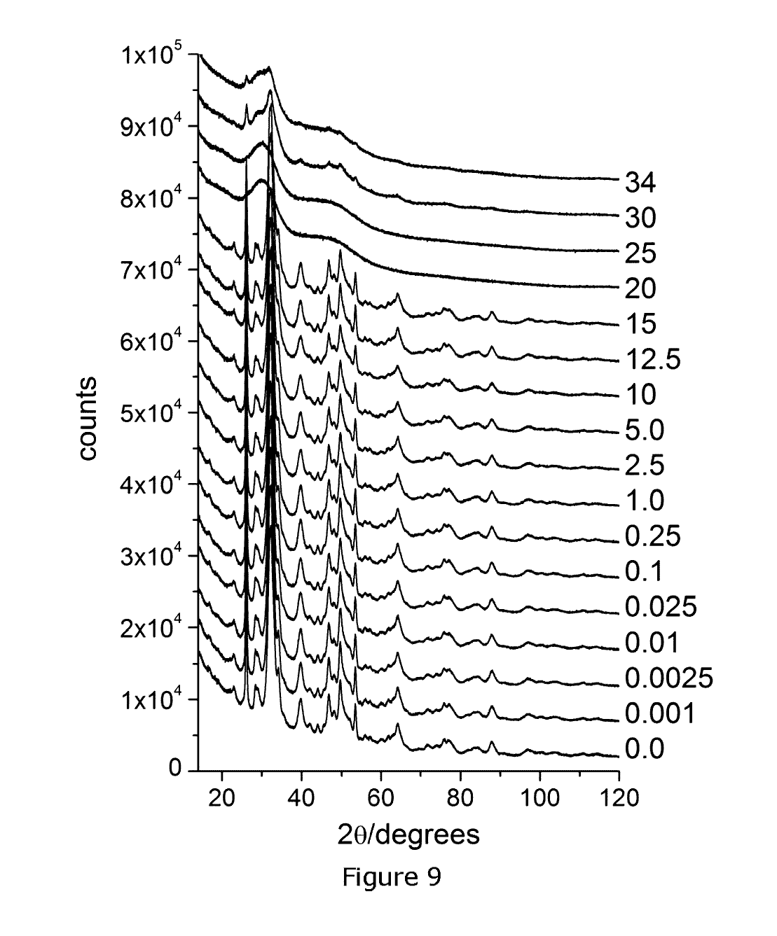

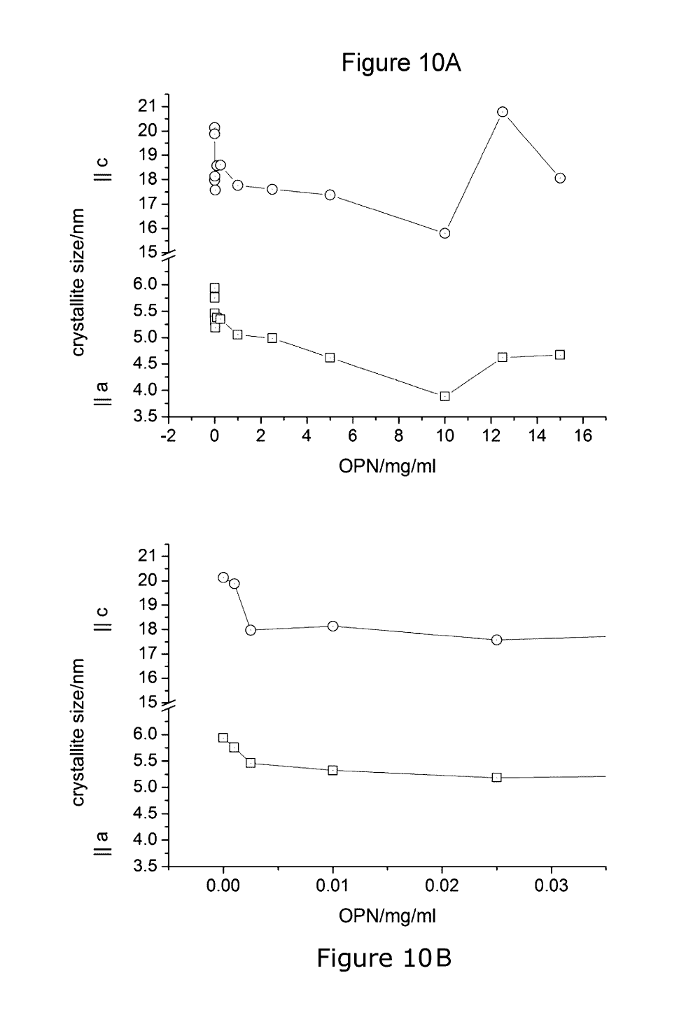

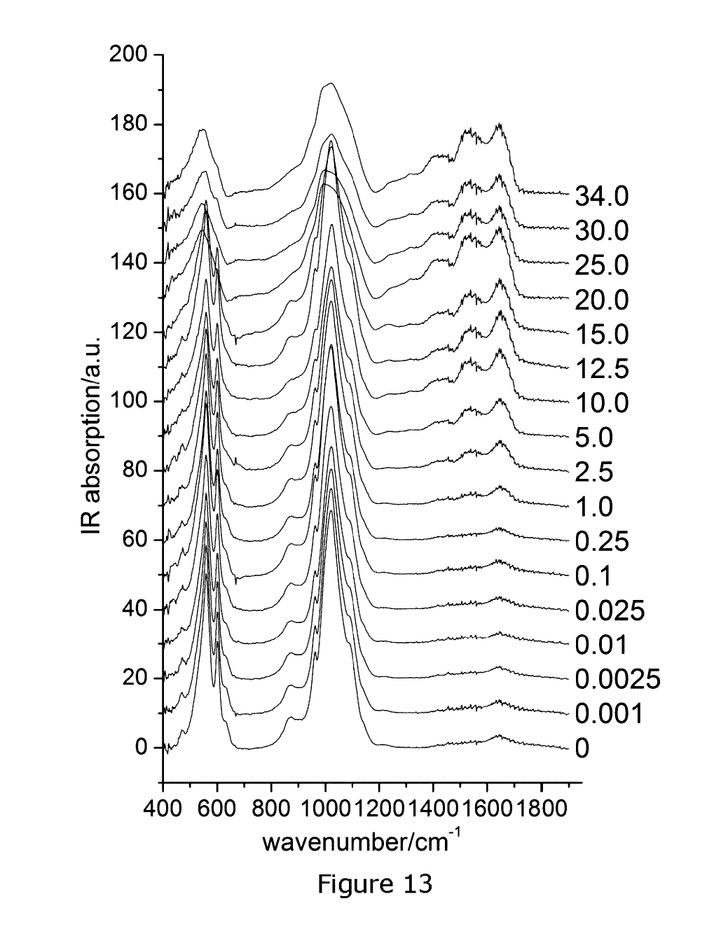

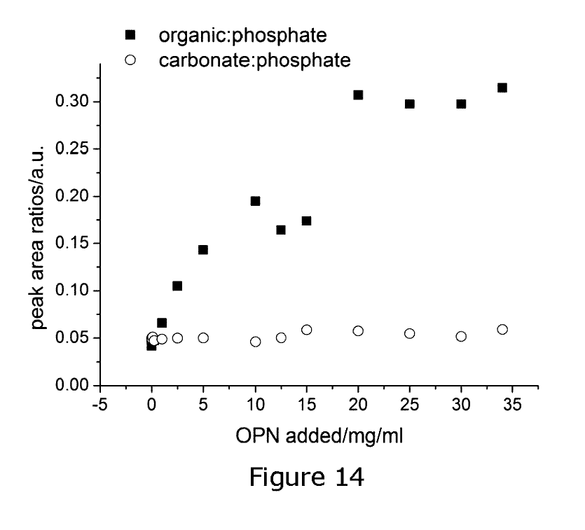

The nanoparticle aggregates were characterized by XRD, FTIR and TGA. For XRD and FTIR measurements, the dry particles were ground to fine powder before measurement. XRD was measured on a Rigaku SmartLab with a Bragg-Brentano setup. Parameters for the measurements were: 6-120° 2θ, step size 0.02, 4°/min. Two scans were performed and averaged for each sample. FIG. 9 shows a selected segment of the combined data. Rietveld refinements were performed for all samples with sufficient crystallinity. Samples with 20 mg/mL or higher OPN content were not refined, as these samples had a high amorphous content which complicates the Rietveld refinements. For the samples which were Rietveld refined, selected parameters were extracted and are presented in FIGS. 10A and 10B. FTIR was conducted on a Nicolet 380 FT-IR, Smart Orbit (Thermo electron corporation). Samples were dried at 60° C. just before measuring. A background was fitted for each individual spectrum and subtracted. The corrected data can be seen in FIG. 13. Integration of specific peaks were made for comparison between phosphate (1200-900 cm⁻¹), carbonate (890-840 cm⁻¹) and organic content (1720-1595 cm⁻¹) of the samples. Comparisons between FTIR mineral:organic and mineral:carbonate peak ratios are shown in FIG. 14.

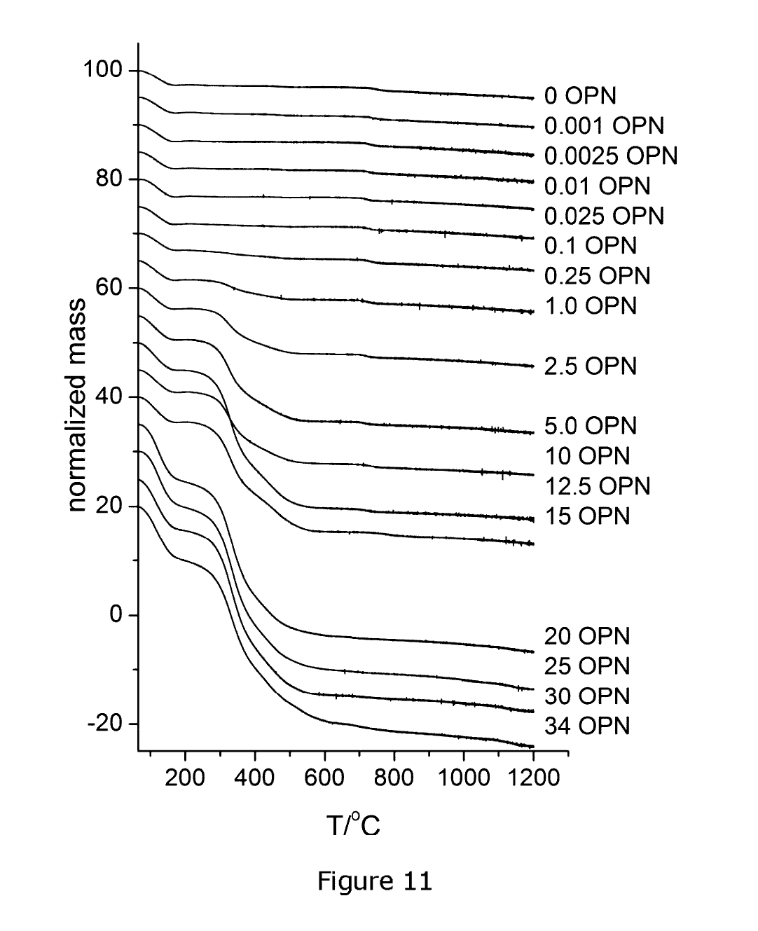

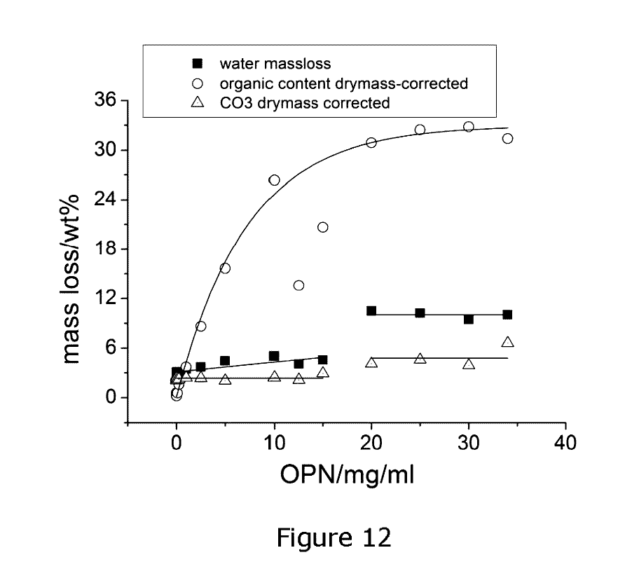

TGA data were recorded on a Netzsch STA 449 C (NETZSCHGeratebau GmbH, Selb, Germany) using an atmosphere of Ar and O₂. TGA data are shown in FIG. 11 while extracted mass losses are shown in FIG. 12.

The XRD data showed that crystalline material formed for all samples with lower than 20 mg/ml OPN. At and above 20 mg/ml a very large amount of amorphous material was observed. At 30 and 34 mg/ml minor peaks were observed atop of the amorphous scattering. The diffraction peaks for the crystalline materials were significantly broader than the experimental resolution reflecting that the crystalline materials are nanocrystalline. The X-ray diffraction data up to and including 15 mg/ml OPN were modelled by Rietveld refinement to extract information on average nanocrystals sizes. The nanocrystals are anisotropic in size and needle shaped with the long morphological axis coinciding with the crystallographic c-axis. Extracted crystallite sizes are shown in FIGS. 10A and 10B. At very low OPN contents, there was a rapid drop in crystallite size in both morphological directions (FIG. 10B) after which the sizes remained almost constant up to an OPN content of ˜1 mg/ml after which it again decreased up to an OPN content of 10 mg/ml. For 12.5 and 15 mg/ml the particle aggregates contained larger nanocrystals than for 10 mg/ml but comparison between background and peak heights showed that there was a large amount of amorphous material in these samples. The content of water, organic components and carbonate was assessed by TGA measurements as shown in FIG. 11. Mass losses are reported in FIG. 12; the mass losses corresponding to organic and carbonate contents were corrected to dry mass (i.e. normalized by mass remaining after the initial loss of water). The water content was in all cases below 15 wt %. It was significantly larger for the amorphous compounds (˜10 wt %) than for the noncrystalline materials. For the nanocrystalline materials, a linearly increasing water content with OPN content was observed following 3.1+0.12·OPN, where OPN is the OPN added in mg/ml. The carbonate content was ˜2.4 dry wt % for the nanocrystalline and 4.8 dry wt % for the amorphous materials, respectively. The organic content increased with OPN added up to 10 mg/ml with 12.5 and 15 mg/ml displaying smaller organic content; at high concentrations the organic content saturated at ˜33 dry wt %. FTIR confirmed the observations above as show in FIGS. 13 and 14.

### Example 2

### Growth of Dental Caries Model Biofilms

The human oral bacterial isolates Streptococcus oralis SK248, Streptococcus downei HG594, Streptococcus mitis SK24, Streptococcus sanguinis SK150 and Actinomyces naeslundii AK6 were used in the experiments. Organisms were cultivated aerobically on blood agar (SSI, Copenhagen, Denmark) and transferred to THB (Roth, Karlsruhe, Germany) at 35° C. until mid to late exponential phase prior to experimental use.

Flow cells (ibiTreat, μ-slide VI, Ibidi, Munich, Germany) were preconditioned with 1/10 THB (pH 7.0). Bacterial cultures, adjusted to an optical density of 0.4 at 550 nm (corresponding to 2-5*10⁹ cells/ml), were inoculated sequentially into the flow channels in the following order: 1. S. oralis SK248; 2. A. naeslundii AK6; 3. S. mitis SK24; 4. S. downei HG594; 5. S. sanguinis SK150. 0.4 ml of each organism was injected through the silicone tubing at the in-port using sterile needles (BD Microlance, 27G, Drogheda, Ireland), and injection holes were sealed with silicone glue (Dow Corning, Wiesbaden, Germany). Following injection, the flow was halted for 30 min to allow for bacterial adhesion. Nonadherent organisms were removed by 10 min of flow prior to injection of the next organism. After inoculation procedure, biofilms were grown for 26 h at 35° C. under constant flow (250 μl/min; 28.3 mm/min) of 1/10 THB (pH 7.0) provided by a peristaltic pump (Watson Marlow 205 U, Wilmington, Mass., USA).

### Example 3

### Binding of OPN to Bacteria in Caries Model Biofilms

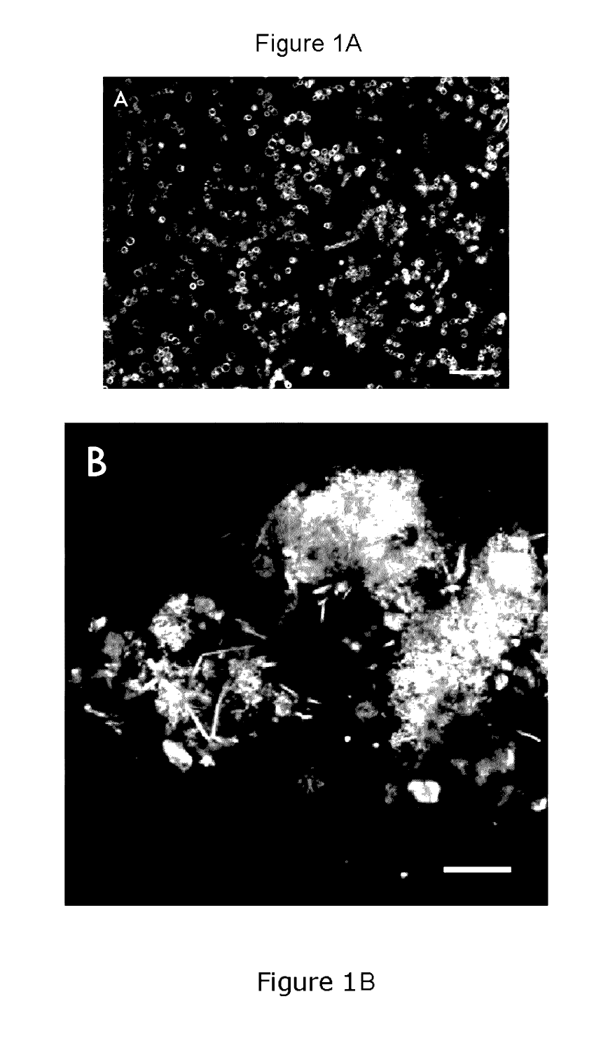

OPN was labelled with fluorescein according to the manufacturer's instructions (Invitrogen, Taastrup, Denmark). After growth phase, biofilms were incubated for 45 min with 100 μl of the labelled protein at 35° C. and imaged using the 488 nm laser line and a 500-550 nm band pass filter. XY resolution was set to 0.1 μm/pixel and Z resolution corresponded to 1 Airy unit (0.8 μm optical slice thickness) (FIG. 1A).

### Example 4

### Binding of Calcium Phosphate Nanoparticle Aggregates Containing OPN to in Vivo Grown Dental Biofilm

In vivo grown dental biofilm was scraped from a tooth surface with a sterile curette and collected on a glass slab (4×4×1 mm). The glass slab was turned upside down, and the biofilm was incubated with calcium phosphate nanoparticle aggregates containing OPN for 30 min at 35° C. After washing with 0.9% NaCl, the biofilm was stained with 30 μM C-SNARF-4 (Sigma-Aldrich, Brøndby, Denmark) that targets both bacteria in the biofilms and nanoparticle aggregates. A Zeiss LSM 510 META (Jena, Germany) with a 63× oil immersion objective, 1.4 numerical aperture (Plan-Apochromat) was used for image acquisition. The probe was exited with a 543 nm laser line (250-300 μW), and fluorescence emission was monitored within 576-608 nm interval (META detector), with the pin hole set to 2 Airy units (1.6 μm optical slice thickness). Images were 364×364 pixels (143×143 μm²) in size and were acquired with pixel dwell time 18 μs, line average 2, 0.4 μm/pixel (zoom 1), 12-bit intensity resolution (FIG. 1B).

### Example 5

### Effect of OPN on Bacterial Growth in Planktonic Culture

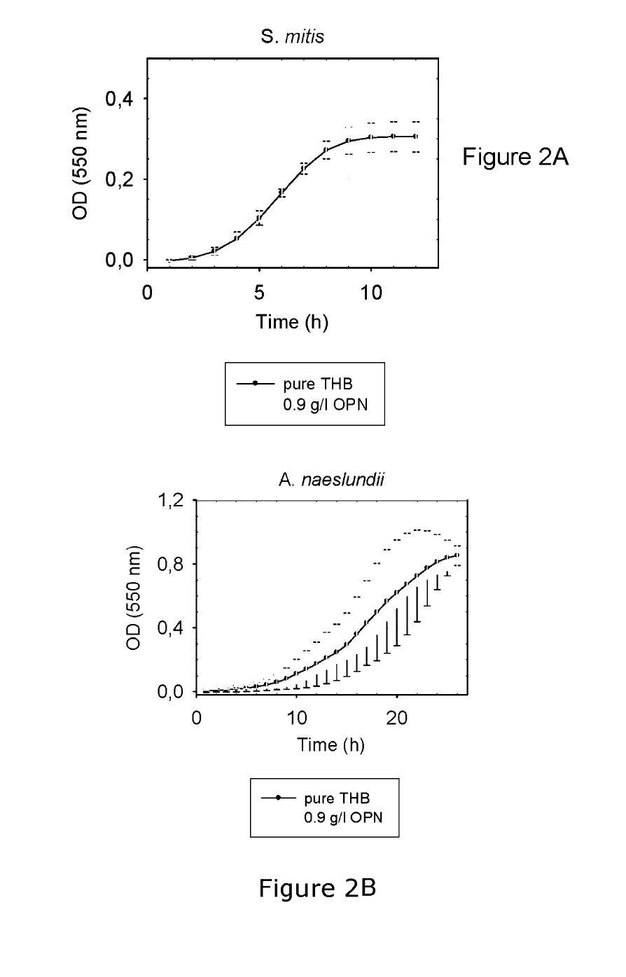

Bacteria were transferred to THB or THB containing 26.5 μmol/l OPN. Aliquots of 100 μl were transferred to a 96 well plate (Sarstedt, Newton, N.C., USA) and OD at 550 nm was measured with a spectrophotometer (BioTek PowerWave XS2, Bad Friedrichshall, Germany). Experiments were carried out in triplicates and repeated once (FIGS. 2A and 2B).

### Example 6

### Effect of Calcium Phosphate Nanoparticle Aggregates Containing OPN on Biofilm Growth in the Caries Model

Biofilms were grown as described above and treated three times with calcium phosphate nanoparticle aggregates containing OPN during growth (3 h, 9 h and 24 h after inoculation procedure). Nanoparticle aggregate suspensions obtained from Example 1 were shaken vigorously, set to settle for 10 min, and 0.4 ml were aspirated from the top of the suspensions. The aspirated suspension had a content of nanoparticle aggregates of approx. 3% (w/v). Then the flow was halted and the aspirated suspension including nanoparticle aggregates was injected into the channels as described for the bacterial inocula. After one hour of incubation the flow was started again. Control treatments were performed in the same way with osteopontin-free calcium phosphate nanoparticle aggregates, silicium dioxide particles of different sizes (100 nm, 500 nm and 2000 nm diameter; 3.5% (w/v) in 0.9% NaCl; Sigma-Aldrich, Brøndby, Denmark), polystyrene particles (1000 nm diameter; 3.5% (w/v) in distilled water; Sigma-Aldrich), and dissolved OPN (0.9 g/l in 0.9% NaCl).

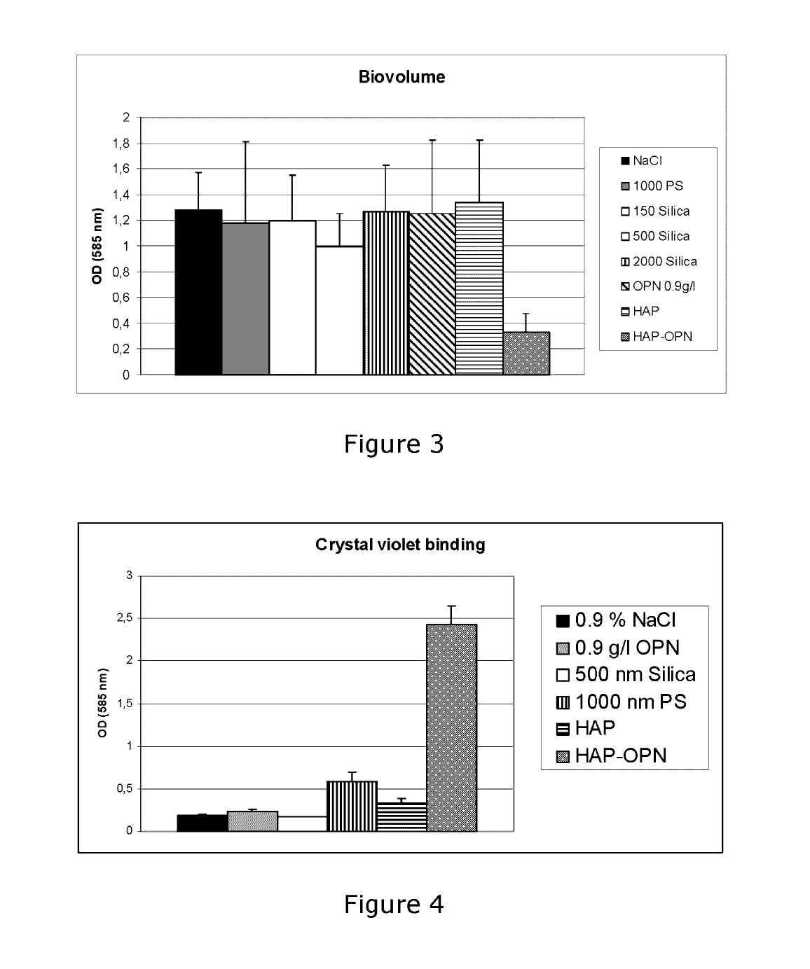

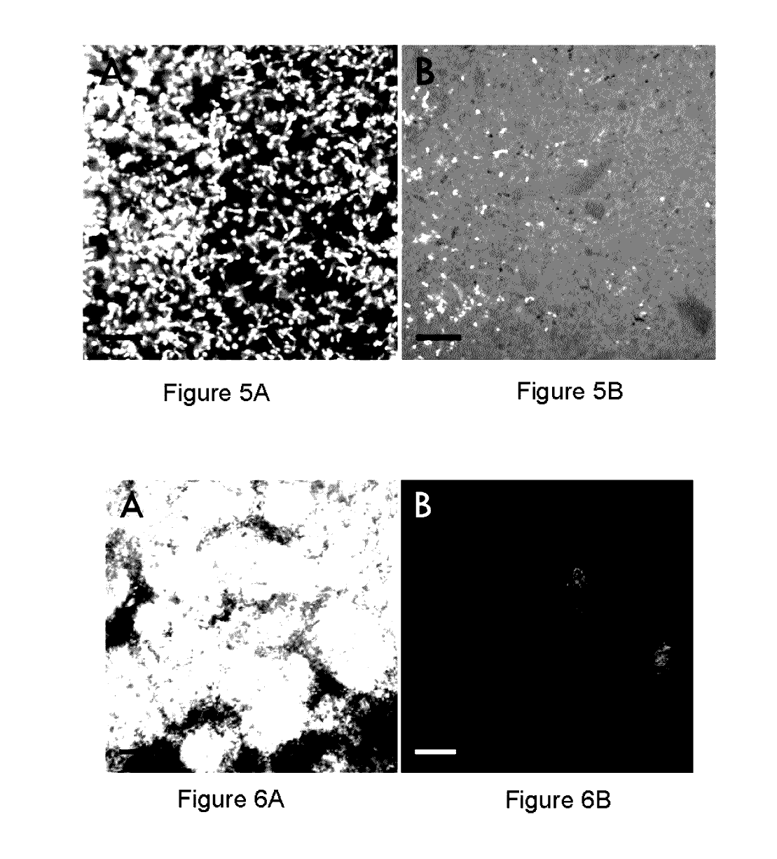

Channels incubated with 0.9% NaCl served as negative controls. Six replicate biofilms were grown for each experimental setting. After biofilm growth, THB was removed from the flow channels by aspiration with paper points. The channels were rinsed with distilled water, dried again and stained with 100 μL of 2% crystal violet solution for 1 h. Then channels were rinsed again with distilled water, dried, and 120 μL of 100% ethanol (Sigma-Aldrich, Brøndby, Denmark) were added during 30 min to destain the biofilms. Thereafter, 100 μL of the stained ethanol solutions, diluted 1:8, were transferred to a 96 well plate (Sarstedt, Newton, N.C., USA), and optical density at 585 nm was measured with a spectrophotometer (BioTek PowerWave XS2, Bad Friedrichshall, Germany). Empty flow channels were processed in the same way and used for background subtraction (FIG. 3).

FIG. 3 shows the effect of different agents on biofilm formation in the caries model, measured by crystal violet staining. Calcium phosphate nanoparticle aggregates containing OPN (HAP-OPN) strongly reduce the amount of biofilm formed in the flow cells, as compared to 1000 nm polystyrene particles, silica particles (150 nm, 500 nm and 2000 nm), OPN-free calcium phosphate particle aggregates and 0.9 g/l OPN.

Thus, Example 6 provides statistically significant evidence demonstrating that one obtains a synergetic anti-biofilm effect by using OPN bound to calcium-containing particles.

### Example 7

### Crystal Violet Binding of Calcium Phosphate Nanoparticle Aggregates Containing OPN

100 μl of calcium phosphate nanoparticle aggregates containing OPN were injected into flow cells (ibiTreat, μ-slide VI, Ibidi, Munich, Germany) and set to dry overnight. Flow channels were stained with crystal violet for 15 min, washed twice with PBS, dried and filled with 120 μl of 100% ethanol for 15 min. 100 μL of the stained ethanol solutions, diluted 1:64, were transferred to a 96 well plate and optical density at 585 nm was measured. Osteopontin-free calcium phosphate nanoparticle aggregates, silicium dioxide particles (500 nm diameter; 3.5% (w/v) in 0.9% NaCl), polystyrene particles (1000 nm diameter; 3.5% (w/v) in distilled water), dissolved OPN (0.9 g/l in 0.9% NaCl) and 0.9 g/I NaCl served as controls. Experiments were performed in triplicates (FIG. 4).

### Example 8

### Effect of Calcium Phosphate Nanoparticle Aggregates Containing OPN on Oral Biofilm Formation In Vivo

Oral biofilms were grown on custom-made glass slabs (4×4×1 mm) (Menzel, Braunschweig, Germany) with a surface roughness of 1200 grit. Glass slabs were mounted recessed in the buccal flanges of individually designed intraoral appliances. Two volunteers kept an appliance intraorally for 72 h, except during tooth brushing, intake of food or liquids other than water and during nanoparticle aggregate dips. One side of the appliance was dipped 5-6 times (30-60 min) each day in a suspension containing 0.9% NaCl and approx. 3% (w/v) nanoparticle aggregates prepared in Example 1. At the same time, the other side of the appliance was dipped in 0.9% NaCl and served as negative control. After 72 h, glass slabs were removed and biofilms were stained with C-SNARF-4 prior to confocal microscopic analysis (FIGS. 6A and 6B).

FIGS. 6A and 6B show that calcium-containing nanoparticle aggregates containing OPN strongly reduce oral biofilm growth in vivo. FIG. 6A: Biofilm grown on a glass slab kept intraorally for 72 h. per day, 5-6 NaCl dips (30-60 minutes) were performed. FIG. 6B: Biofilm grown on a glass slab kept intraorally for 72 h.

Thus, Example 8 confirms that the anti-biofilm effect observed in Example 6 also exists in vivo.

### Example 9

### Effect of Calcium Phosphate Nanoparticle Aggregates Containing OPN on pH of Planktonic Bacterial Cultures

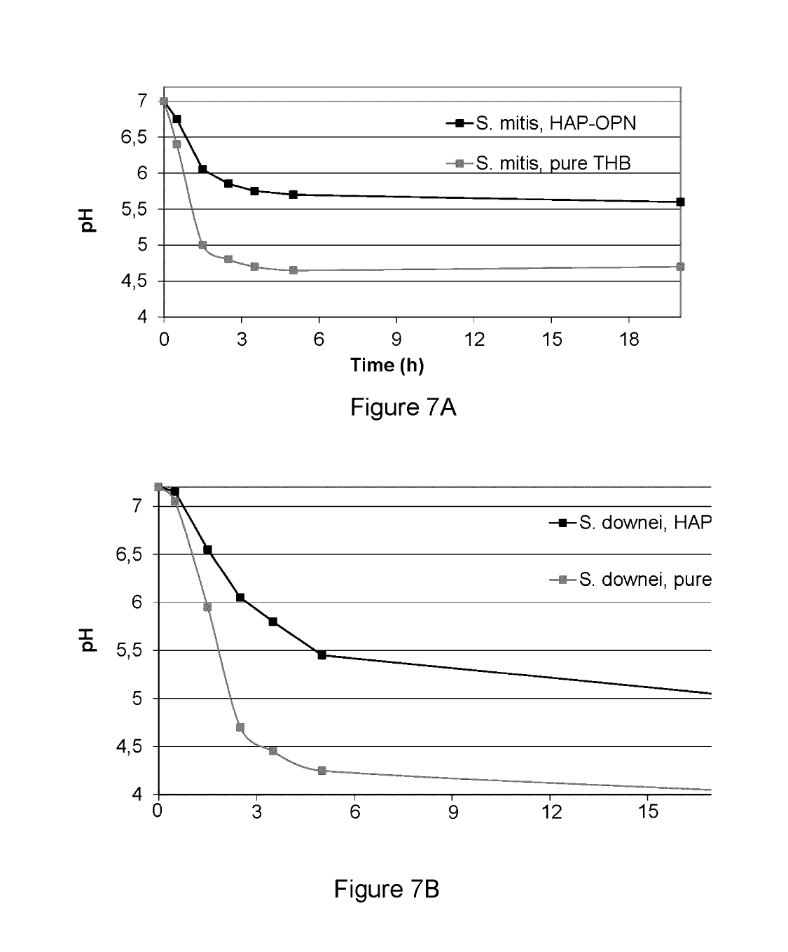

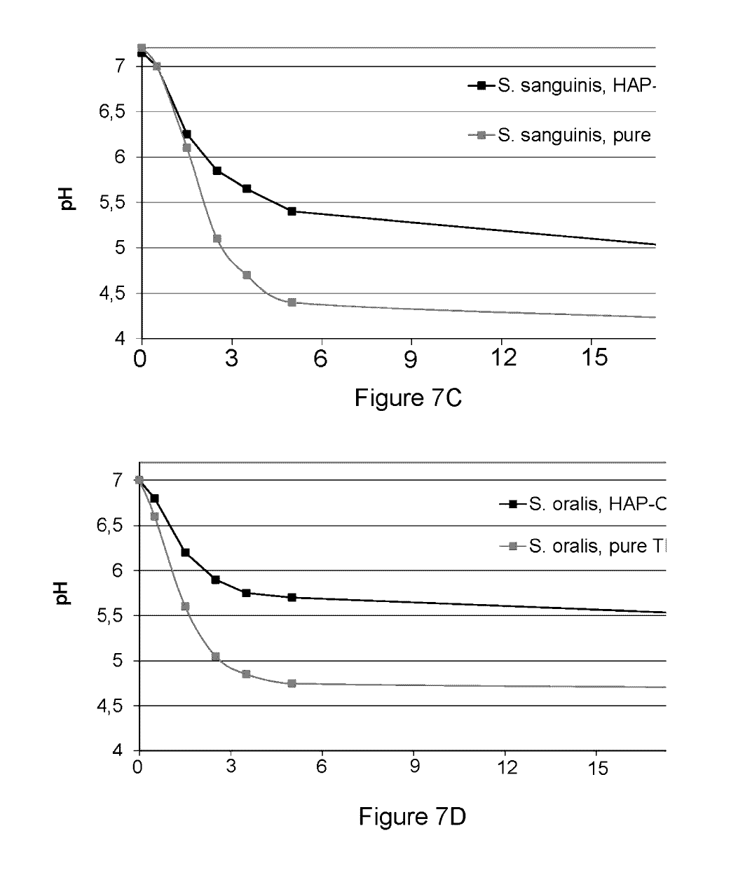

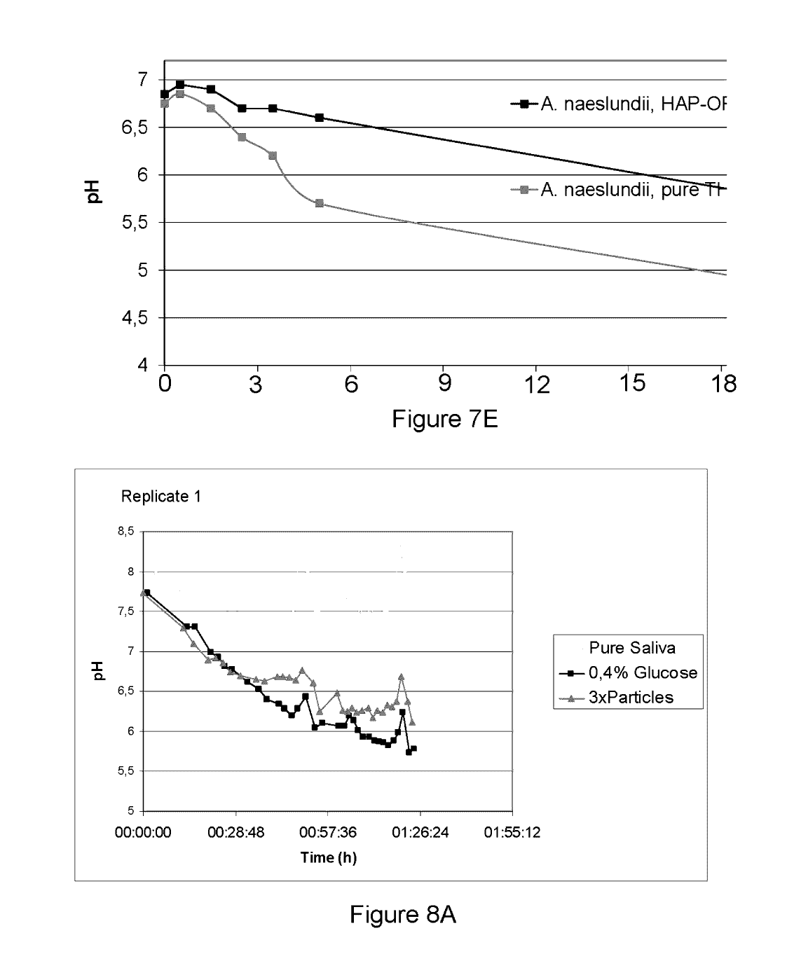

Bacterial cultures were grown in THB until mid-exponential phase, washed twice and transferred to sterile saliva. OD was adjusted to 1.0 (550 nm), 0.4% glucose (w/w) was added and 1 ml of bacterial suspension was mixed with 1 ml of calcium phosphate nanoparticle aggregates containing OPN or 1 ml of 0.9% NaCl. pH was measured for 20 h. Experiments were performed in duplicate and repeated once (FIGS. 7A-7E).

### Example 10

### Effect of Calcium Phosphate Nanoparticle Aggregates Containing OPN on DH in Caries Model Biofilms

Inoculation procedure was carried out as described above. Biofilms were then grown on 1/10 diluted THB for 8 h at 35° C. with a flow rate of 250 μL/min. By that time, stable thin biofilms had formed. Then the medium was changed to carbohydrate free beef extract (Scharlau, Barcelona, Spain) and the flow rate was reduced to 50 μL/min to minimize shear forces in the flow cell. 1 h, 12 h and 17 h after medium change, HAP-OPN particles were injected and incubated with the biofilms for 1 h as described above. Control channels were incubated with 0.9% NaCl.

Confocal microscopic calibration of the ratiometric pH-sensitive probe C-SNARF-4: HEPES buffer solutions (50 mM; adjusted to pH 4.5-8.5 in steps of 0.1 pH units), containing C-SNARF-4 at a concentration of 30 μM, were imaged in flow channels. A Zeiss LSM 510 META (Jena, Germany) with a 63× oil immersion objective, 1.4 numerical aperture (Plan-Apochromat) was used for image acquisition. The probe was exited with a 543 nm laser line (250-300 μW), and fluorescence emission was monitored simultaneously within 576-608 nm (green) and 629-661 nm (red) intervals (META detector), with the pin hole set to 2 Airy units (1.6 μm optical slice thickness). Images were 364×364 pixels (143×143 μm²) in size and were acquired with pixel dwell time 18 μs, line average 2, 0.4 μm/pixel (zoom 1), 12-bit intensity resolution. For every third pH value, a measurement was performed on unstained HEPES buffer (50 mM, pH 8.5) for background subtraction. Additionally, unstained solutions of glucose (20% w/v) and lactate (20% w/v), 1/10 diluted THB (pH 7), sterile saliva, PBS (pH 7.4, Sigma-Aldrich, Brøndby, Denmark), untreated biofilms and HAP-OPN particle suspensions were imaged with identical microscope and laser settings. No autofluorescent signals were emitted by any of these controls in the wavelength ranges 576-608 nm and 629-661 nm.

For ratio calculation, regions of 100×100 pixels were defined within each image and the average and standard deviations were determined using the LSM acquisition software. Subsequently, the ratio R, standard deviation sp and standard error of mean, S_{R}, were calculated for each pH value according to equations (1), (2) and (3):

$$\begin{matrix}R = \frac{g - {b}_{g}}{r - {b}_{r}} & ( 1 ) \\ {s}_{R} = \frac{1}{{( {( R - {b}_{r} )}^{2} )}^{1 / 2}} {( {s}_{g}^{2} + {s}_{bg}^{2} + \frac{{( g - {b}_{g} )}^{2}}{{( r - {b}_{r} )}^{2}} ( {s}_{r}^{2} + {s}_{br}^{2} ) )}^{1 / 2} & ( 2 ) \\ {S}_{R} = \frac{1}{{( {100}^{2} )}^{1 / 2}} {( {s}_{r}^{2} )}^{1 / 2} & ( 3 )\end{matrix}$$

g, r, s_{g} and sᵣ are the averages and standard deviations within the 100×100 pixels region defined in the respective green and red images. b_{g}, bᵣ, s_{bg} and s_{br} are the corresponding values for the background images. The resulting values of R were plotted in MATLAB (MathWorks, Natick, Mass., US), and fitted to the function:

$$pH = ln ( \frac{- 1.6546}{R - 1.47469} - 1 ) \frac{1}{( 2.3981 )} + 59799 \qquad ( 4 )$$

Measurements were performed twice and proved to be highly reproducible.

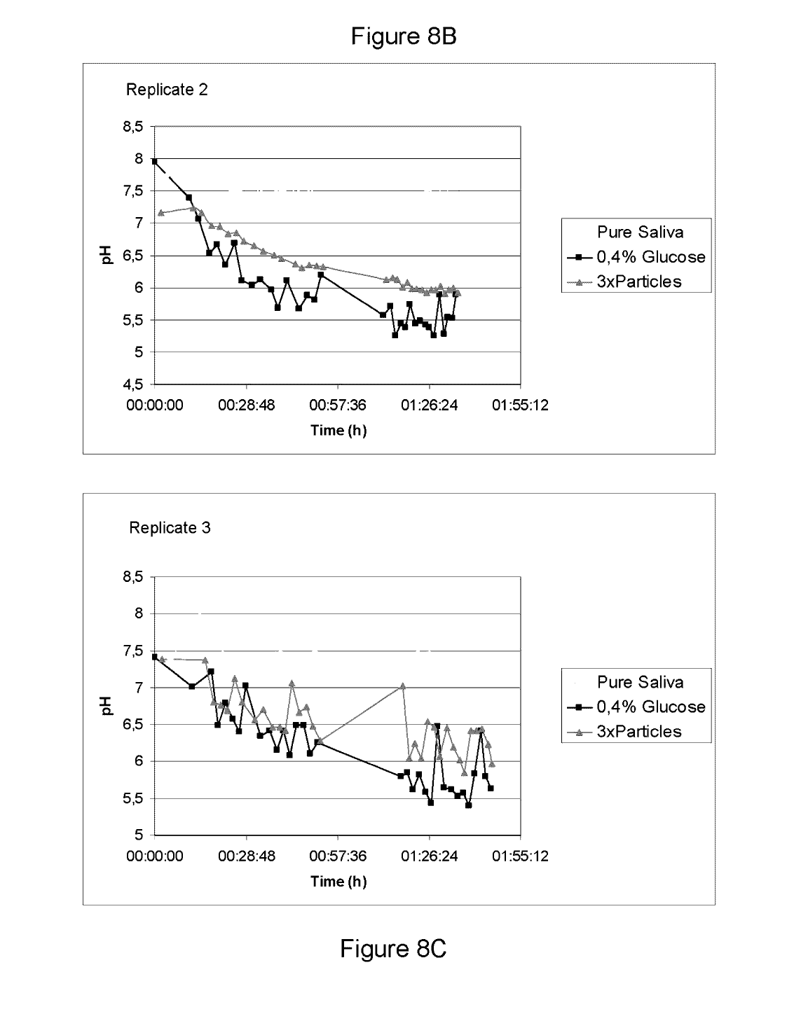

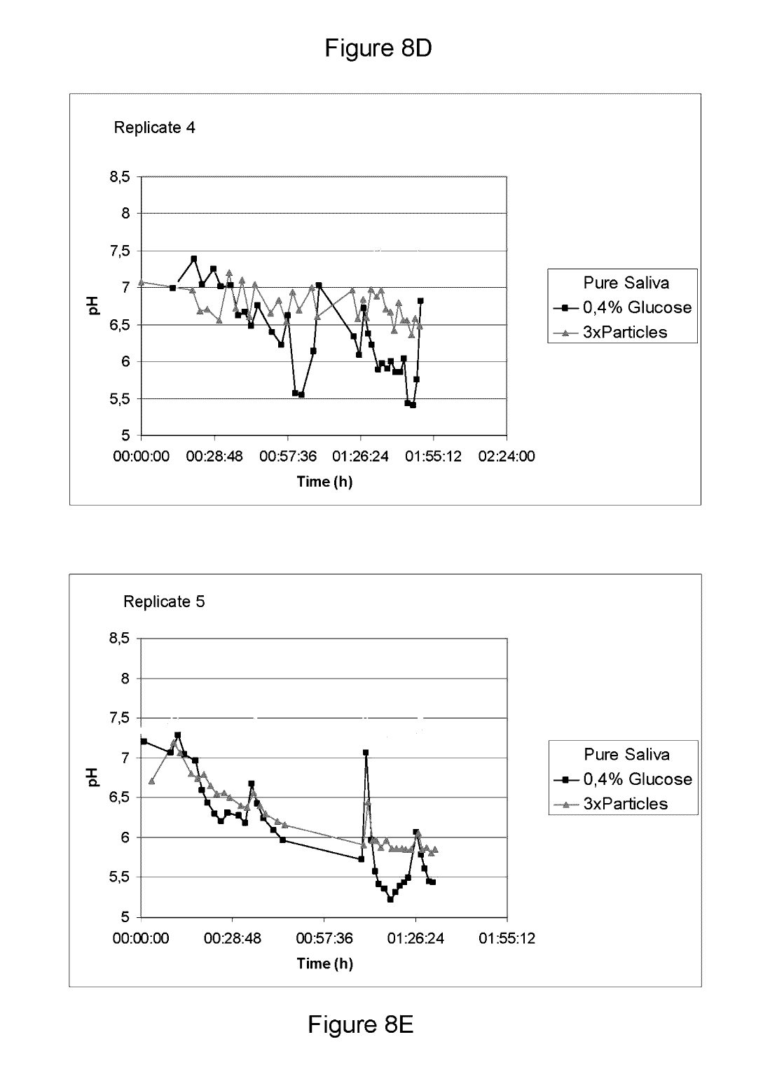

Biofilm pH imaging: Three biofilms were grown in parallel, one of which was treated with calcium phosphate nanoparticle aggregates containing OPN as described above. Thereafter, biofilms were washed twice with sterile saliva, and C-SNARF-4 was added to a concentration of 30 μM. The flow cell was transferred to the microscope, which was kept at 37° C. with an XL incubator (PeCon, Erbach, Germany), and baseline pH images were acquired in the bottommost layer of the biofilms. Subsequently, glucose-free saliva was replaced by salivary solution containing 0.4% (w/v) glucose and 30 μM of C-SNARF-4 in two of the three channels. In each of the three biofilms, pH images were acquired in 16 microscopic fields of view chosen at random. XY positions were marked in the LSM software, and 60 min after the addition of glucose, identical microscopic fields were imaged again in the same order. For background subtraction, images were acquired with the 543 nm laser switched off. The microscope and laser settings were identical to those of the calibration measurements. Five independent replicates of the experiments were performed (FIGS. 8A-8E).

1-24. (canceled)

25. Nanoparticle aggregates comprising a) osteopontin (OPN) and b) a first particle comprising calcium and/or strontium for curing, alleviating and/or preventing a biofilm-related disease.

26. Nanoparticle aggregates according to claim 25, wherein the biofilm-related disease involves a bacterial infection.

27. Nanoparticle aggregates according to claim 25, wherein the biofilm-related disease is an oral disease.

28. Nanoparticle aggregates according to claim 27, wherein the biofilm-related disease is dental caries.

29. Nanoparticle aggregates according to claim 27, wherein the biofilm-related disease is gingivitis.

30. Nanoparticle aggregates according to claim 27, wherein the biofilm-related disease is periodontitis.

31. Nanoparticle aggregates according to claim 25, wherein the biofilm-related disease is a disease selected from the group consisting of bacterial endocarditis, chronic wound infections, implant infections, otitis media, cystic fibrosis, and a combination thereof.

32. Nanoparticle aggregates according to claim 25 for curing, alleviating and/or preventing a bacterial infection, e.g. a bacterial wound infection.

33. Nanoparticle aggregates comprising a) OPN and b) a first particle comprising calcium and/or strontium for reducing or preventing microbial biofilm growth or for removing microbial biofilm.

34. Nanoparticle aggregates according to claim 33, wherein the biofilm is dental plaque.

35. Nanoparticle aggregates according to claim 33, wherein the biofilm contains bacteria having a OPN binding capacity of at least 50 OPN molecules per cell.

36. Nanoparticle aggregates according to claim 25, wherein the biofilm contains, or even consists of, one or more bacteria selected from the group consisting of Streptococcus spp., Staphylococcus spp., Pseudomonas spp. Actinomyces spp., Lactobacillus spp., Aggregatibacter spp., Bacteroides spp., Listeria spp., Campylobacter spp., Eikenella spp., Porphyromonas spp., Prevotella spp., Treponema spp., and combinations thereof.

37. Nanoparticle aggregates according to claim 25, wherein the first particle comprises calcium.

38. Nanoparticle aggregates according to claim 25, wherein the first particle comprises strontium.

39. Nanoparticle aggregates according to claim 25, wherein the first particle comprises calcium and strontium.

40. Nanoparticle aggregates according to claim 25, wherein the first particle comprises an inorganic salt of calcium and/or strontium.

41. Nanoparticle aggregates according to claim 40, wherein the inorganic salt comprises a phosphate species, sulfate, and/or carbonate.

42. Nanoparticle aggregates according to claim 25, wherein the first particle comprises at least 50% (w/w) calcium phosphate.

43. Nanoparticle aggregates according to claim 25, wherein the first particle is capable of releasing calcium and/or strontium.

44. Nanoparticle aggregates according to claim 25, wherein the first particle is a nanoparticle.

45. Nanoparticle aggregates according to claim 25, comprising a second particle of the same type as the first particle.

46. Nanoparticle aggregates according to claim 25, having a hydrodynamic radius of at most 5 micron.

47. Use of nanoparticle aggregates comprising a) OPN and b) a first particle comprising calcium and/or strontium, for reducing or preventing microbial biofilm growth or for removing microbial biofilm.

48. Use according to claim 47, wherein the biofilm contains, or even consists of, one or more bacteria selected from the group consisting of Streptococcus spp., Staphylococcus spp., Pseudomonas spp. Actinomyces spp., Lactobacillus spp., Aggregatibacter spp., Bacteroides spp., Listeria spp., Campylobacter spp., Eikenella spp., Porphyromonas spp., Prevotella spp., Treponema spp., and combinations thereof.
